(19) United States
(12) Patent Application Publication
(10) Pub. No.: US 2025/0302975 A1
(43) Pub. Date: Oct. 2, 2025

(54) METHODS AND COMPOSITIONS FOR AGONISING TLR3

(71) Applicant: The Regents of the University of California, Oakland, CA (US)

(72) Inventors: Nicole F. Steinmetz, San Diego, CA (US); Eunkyeong Jung, La Jolla, CA (US)

(21) Appl. No.: 19/092,592

(22) Filed: Mar. 27, 2025

Related U.S. Application Data
(60) Provisional application No. 63/572,162, filed on Mar. 29, 2024.

(51) Int. Cl. A61K 47/64

(52) U.S. Cl. CPC A61K 47/6425

(57) ABSTRACT

Provided herein are compositions and methods for inhibiting the growth of a mammalian cancer cell growth or stimulating the immune response of a mammal, by contacting the cell or administering to the mammal an effective amount of a viral nanoparticle comprising at least one TLR agonist and a chemotherapeutic agent.

### CROSS-REFERENCE TO RELATED APPLICATIONS

This application claims the benefit of priority to U.S. Provisional Patent Application No. 63/572,162, filed on Mar. 29, 2024, the contents of which are incorporated herein by reference in their entirety.

### STATEMENT OF GOVERNMENT SUPPORT

This invention was made with government support under Grant Nos. R01 CA224605, R01 CA253615, and R01 CA274640, awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Mar. 25, 2025, is named 114198-6501_SL.xml and is 5,178 bytes in size.

### BACKGROUND

Cancer immunotherapy is a promising collection of treatments that harness the ability of the immune system to fight cancer. In contrast to surgery, radiotherapy and chemotherapy, each of which requires a radical external intervention, immunotherapy is based on enhancing natural anti-cancer mechanisms, particularly those that are suppressed by aggressive tumors [1]. Cancer cells produce tumor-associated and neoantigens that are recognized by the immune system, allowing the tumors to be targeted and eradicated by tumor-infiltrating lymphocytes (TILs). However, many cancer cells also possess the ability to create an immunosuppressive tumor microenvironment (TME) by blocking the infiltration, activation and effector functions of immune cells, evading immunosurveillance, and stimulating pathways that recruit immunosuppressive regulatory T (T_{reg}) cells and tumor-associated macrophages [2]. The immunosuppressive TME results in so-called “cold” tumors that tend to respond poorly to immunotherapy. The administration of systemic immunomodulators such as cytokines and checkpoint inhibitors can reverse this immunosuppressive state, but they cause severe side effects [3].

### SUMMARY OF THE DISCLOSURE

One approach to overcome these challenges is intratumoral therapy, in which a therapeutic is introduced directly into the tumor to induce a local, antigen-specific anti-tumor immune response [4]. The therapeutic agent is generally an immunostimulatory adjuvant, which converts the immunosuppressive TME to an immunostimulated phenotype, thus restoring the normal cancer immunity cycle and allowing the natural immune response to deal with “hot” tumors [5, 6]. Immunologically cold tumors can be made hot by targeting endosomal Toll-like receptors (TLRs), specifically TLR3, TLR7, TLR8 and TLR9, within antigen-presenting cells (APCs) [7]. In the clinic, the dermal application of imiquimod (TLR7 agonist) and resiquimod (TLR7/8 agonist) has confirmed the ability of such drugs to induce a local immune response against cutaneous tumors while limiting systemic exposure and side effects [8], and the approval of tamilogene laherparepvec (T-VEC), an oncolytic herpesvirus that expresses granulocyte-macrophage colony-stimulating factor (GM-CSF), has confirmed that intratumoral immunotherapy is suitable for the treatment of melanoma [9].

Applicant focused polyinosinic:polycytidylic acid (poly(I:C)), which resembles the structure of dsRNA and agonizes TLR3. TLR3 recognizes double stranded RNA to activate innate immune cells (macrophages, dendritic cells) to become APCs leading to CD4⁺ T cell responses to switch from Th2 to Th1 while boosting the CD8⁺ T cell response and inhibiting T_{reg} cells [10]. TLRs are activated by a variety of synthetic agonists, including polyinosinic:polycytidylic acid (poly(I:C)), which resembles the structure of dsRNA and interacts with TLR3. The intratumoral application of free poly(I:C) is hampered by rapid washout effects and limited cell uptake—to overcome these shortcomings and control tissue diffusivity and enhance cell uptake, Applicant showed that the PVX nanoparticles have immunomodulatory effects after intratumoral administration and combination of PVX and chemotherapy doxorubicin resulted in potent anti-tumor immunity. This supports earlier studies showing that immunotherapy and chemotherapy can achieve synergistic effects that benefit patient outcomes. The potent effect of PapMV was attributed to its encapsulated RNA, which was shown to activate signaling through TLR7 [14]. Further, Applicant found that CPMV is a potent anti-tumor agent and its potency is attributed to its signaling through multiple TLRs; the proteinaceous capsid is recognized by TLR2 and TLR4 while the RNA is recognized by TLR7 [15, 16]. In contrast, CCMV alone did not exhibit anti-tumor efficacy; however, when loaded with CpG oligodeoxynucleotides it was shown to induce the activity of tumor-associated macrophages resulting in potent anti-tumor immune responses in tumor mouse models [17]. Given that CCMV has proven useful for the delivery of TLR agonists but was not tested in the context of combination therapy, here Applicant developed CCMV particles loaded with the TLR3 agonist poly(I:C) and combined it with oxaliplatin, an antineoplastic drug [18]. Without being bound by theory, it is believed that the chemotherapy induces cancer cell death to release tumor associated and neoantigens to be processed by innate immune cells recruited and activated by the TLR3 agonist, therefore resulting in improved therapy success. This was tested in a mouse model of colon cancer. Applicant also provides the underlying immunological mechanism tough a combination of chemo/cytokine analysis, immunological cell profiling, and tumor histology imaging.

Provided herein are compositions and methods of use for nanoparticles in combination with a chemotherapeutic agent. In some embodiments, the nanoparticles are plant viral nanoparticles or bacteriophage virus-like particles (VLP). The nanoparticles comprise at least one Toll-like Receptor (TLR) agonist that activates a TLR. In some embodiments, the TLR agonist comprises, or consists essentially of, or yet further consists of a double-stranded RNA (dsRNA) or synthetic dsRNA, for example polyinosinic acid and polycytidylic acid (poly(I:C)). In other embodiments, the TLR agonist comprises, or consists essentially of, or yet further consists of 2-methoxyethoxy-8-oxo-9-(4-carboxybenzyl)adenine (1V209). In some aspects the nanoparticle and a chemotherapeutic agent are combined, and the combination is used to treat or prevent cancer or a tumor. In other embodiments, the viral nanoparticle and chemotherapeutic agent are used to treat or prevent cancer or a tumor, and the viral nanoparticle and chemotherapeutic agent are applied concurrently or separately. In some embodiments the viral nanoparticle and chemotherapeutic agent are applied as part of one dose, or in at least two separate doses.

In particular, the TLR agonists of interest in the compositions and methods disclosed and described herein are TLR3 agonist poly(I:C) and TLR7 agonist 1V209. In one aspect, the chemotherapeutic agent in the compositions and methods of use is oxaliplatin.

Applicant demonstrates herein the synergistic effect demonstrated by a combination of a viral nanoparticle comprising a TLR agonist and a chemotherapeutic agent in inhibiting the proliferation of cancer cells and controlling cancer cells as compared to the individual components.

Further provided are kits containing the viral nanoparticle and TLR agonist and optionally, instructions for use.

### BRIEF DESCRIPTION OF THE FIGURES

FIGS. 1A-1E: Characterization of CCMV-Poly(I:C) VLPs. (FIG. 1A) Schematic illustration of preparation of CCMV-Poly(I:C) VLPs. CCMV was disassembled into capsid proteins (CPs) at pH 7.5 and high ionic condition (Ca2+, Mg2+). Disassembled CPs were resembled with Poly(I:C) at pH 4.2. (FIG. 1B) UV-Vis absorption spectra of CCMV and CCMV-Poly(I:C). Inset table shows the absorbance ratio at 260 and 280 nm. (FIG. 1C) Agarose gel electrophoresis. Left panel is an agarose gel image of RNA in 2% GelRed visualized under UV light and right panel is a protein image visualized under white light after staining with Coomassie Brilliant Blue. (FIG. 1D) TEM images of CCMV and CCMV-Poly(I:C) (Scale bar=200 nm). Average size of particles was measured using an ImageJ software. Data are means±s.d (n=5). (FIG. 1E) SEC profiles of CCMV and CCMV-Poly(I:C).

FIG. 2: Immunogenicity activity of CCMV-Poly(I:C) VLPs. Activation of RAW-Blue cells by treatment of CCMV, Poly(I:C), and CCMV-Poly(I:C). LPS served as a positive control and PBS served as a negative control. Values are mean±s.d (n=7). Statistical significance was determined using one-way ANOVA test with a post hoc Tukey's HSD test (***P<0.001).

FIGS. 3A-3E: Enhanced therapeutic efficacy of CCMV-poly(I:C) VLPs plus oxaliplatin in mice with CT26 tumors induced by i.p. inoculation. (FIG. 3A) Schematic illustration of the experimental timeline. CT26 cells were inoculated i.p. into female BALB/c mice (n=7 per group). The tumor-bearing mice were then treated i.p. with CCMV-poly(I:C) and/or oxPt as well as the separate CCMV and poly(I:C) components on days 3, 10 and 17. Gray arrows indicate the treatment days. (FIG. 3B) Tumor growth curves. Data are means±standard deviations (n=7). Statistical significance was determined by one-way ANOVA with a post hoc Tukey's HSD test (***p<0.001, **p<0.01). (FIG. 3C) Tumor growth kinetics of individual mice. (FIG. 3D) Survival rates of mice. (FIG. 3E) Body weights of mice. Mouse image from BioRender.com.

FIGS. 4A-4D: Enhanced therapeutic efficacy of CCMV-Poly(I:C) VLPs with oxaliplatin in mice bearing s.c. CT26 tumors. (FIG. 4A) Schematic illustration of experimental timeline. CT26 cells were inoculated s.c. into left flank of BALB/c mice (8 mice/group). The tumor bearing mice were i.t. treated with CCMV-Poly(I:C) and oxaliplatin (Oxal) once the tumor sizes reached ˜30 mm3. Gray arrows indicate the treatment days. (FIG. 4B) Tumor growth curves. Data are means±standard deviations (n=8). The results were compared by one-way ANOVA with a post hoc Tukey's HSD test (***p<0.001, **p<0.01). (FIG. 4C) Tumor growth kinetics of individual mice. (FIG. 4D) Survival curves.

FIGS. 5A-5B: Stimulation of cytokine production by CCMV-poly(I:C) VLPs with oxaliplatin in mice bearing CT26 tumors induced by i.p. inoculation. (FIG. 5A) Experimental timeline for treatment and the collection of i.p. washes. (FIG. 5B) The level of pro-inflammatory cytokines and chemokines measured using a mesoscale discovery (MSD) assay. Data are means±standard deviations (n=6). Statistical significance was determined by one-way ANOVA with a post hoc Tukey's HSD test (***p<0.001, **p<0.01, *p<0.05). Mouse image from BioRender.com.

FIGS. 6A-6C: Immunogenicity of CCMV-poly(I:C) VLPs with oxaliplatin in mice bearing CT26 tumors induced by i.p. inoculation. (FIG. 6A) Experimental timeline for treatment and the collection of i.p. washes and spleens on day 24. (FIG. 6B) The population of CD4+ and CD8+ cells in the i.p. washes. (FIG. 6C) The level of IL-4 and IFN-γ produced by splenocytes from immunized mice. Data are means±standard deviations (n=6). Statistical significance was determined by one-way ANOVA with a post hoc Tukey's HSD test (***p<0.001, **p<0.01, *p<0.05). Mouse image from BioRender.com.

FIGS. 7A-7E: Histological evaluation of colon tumor tissues treated with CCMV-poly(I:C) VLPs and oxPt. (FIG. 7A) Experimental timeline. Gray arrows indicate treatment with CCMV-poly(I:C) VLPs and oxPt. CT26 s.c. tumors were harvested on day 24. (FIG. 7B) TUNEL staining of tumor tissues (scale bar=20 m). (FIG. 7C) Quantitative analysis of fluorescence from TUNEL-positive cells using ImageJ software. (FIG. 7D) Calreticulin (CRT) exposure staining and (FIG. 7E) quantification of fluorescence intensity. Data are means±standard deviations (n=4). Statistical significance was determined by one-way ANOVA with a post hoc Tukey's HSD test (***p<0.001, **p<0.01, *p<0.05). Mouse image from BioRender.com.

FIGS. 8A-8B: Characterization of CCMV capsid protein (CP). (FIG. 8A) TEM image of CCMV CP. Scale bar is 200 nm. (FIG. 8B) SEC profile of CCMV CP.

FIG. 9: Stimulation of HEK293 cells with CCMV-Poly(I:C) VLPs.

FIGS. 10A-10D: In vivo therapeutic efficacy of CCMV-Poly(I:C) VLPs on B16F10 melanoma. (FIG. 10A) Experimental timeline; C57BL6 mice were i.p. challenged with CCMV-Poly(I:C) once the i.d. B16F10 tumor reaches ˜30 mm3 (5-7 mice/group). Grey arrows indicate the treatment days. (FIG. 10B) Tumor growth curves. (FIG. 10C) Mice survival rate. (FIG. 10D) Individual tumor growth kinetics for each group.

FIG. 11: The synthesis of CPMV−1V209, CCMV−1V209 and Qβ−1V209 particles. EDC/NHS chemistry was used, and the resulting conjugation efficiency was determined by UV-vis absorbance spectrophotometry. Data are means±standard deviation (n=3). EDC—1-ethyl-3-(3-dimethylaminopropyl)carbodiimide, NHS—N-hydroxysuccinimide.

FIGS. 12A-12D: Characterization of CPMV−1V209, CCMV−1V209 and Qβ−1V209 particles. (FIG. 12A) Analysis of 1V209-conjugated and native particles by 0.8% (w/v) agarose gel electrophoresis. Gels were stained with GelRed and visualized under UV light (left panel, RNA detection) and stained with Coomassie Brilliant Blue and visualized under white light (right panel, protein detection). The conjugation drives the migration from the cathode (−, top) to toward the anode (+, bottom). (FIG. 12B) Representative TEM images (scale bar=200 nm). (FIG. 12C) Size distribution of particles determined by DLS. The boxed insets show the average diameter (D, nm) and polydispersity (PDI) of the particles. (FIG. 12D) SEC profiles of the conjugated particles. The boxed insets show the elution volume (V, mL) and the absorbance ratio at 260 and 280 nm (A260/280).

FIGS. 13A-13B: The immunogenicity of 1V209 is enhanced by conjugation to CPMV, CCMV and Qβ. (FIG. 13A) Evaluation of NF-κB/AP-1 activation in RAW-Blue cells. (FIG. 13B) IL-6 levels measured by ELISA. Admixture of VNPs/VLPs and 1V209 is named as VNPs/VLPs+1V209 and conjugation of VNPs/VLPs and 1V209 is named as VNPs/VLPs-1V209. Data are means±SEM (n=6). The results were compared by one-way ANOVA with Tukey's multiple comparisons test (***P<0.001, **P<0.01, *P<0.05).

FIGS. 14A-14D: Therapeutic effects of CPMV−1V209, CCMV−1V209 and Qβ−1V209 particles in the B16F10 melanoma model in vivo. (FIG. 14A) Timeline of B16F10 cell inoculation and intratumoral (i.t.) particle injections. (FIG. 14B) Tumor growth curves. Blue arrows indicate the treatment days. (FIG. 14C) Volumetric scatter plot of individual tumors on day 28. Data are means±SEM (n=5). The results were compared by one-way ANOVA with Tukey's multiple comparisons test (***P<0.001, **P<0.01). (FIG. 14D) Survival rates of tumor-bearing mice over 70 days (dashed lines show the intersection with median survival for each treatment). The results were compared using the log-rank (Mantel-Cox) test (*P<0.05).

FIGS. 15A-15E: Therapeutic effects of CPMV−1V209, CCMV−1V209 and Qβ−1V209 particles in the CT26 colon cancer model in vivo. (FIG. 15A) Timeline of CT26 cell inoculation and treatment. (FIG. 15B) Tumor growth curves. Blue arrows indicate the treatment days. (FIG. 15C) Volumetric scatter plot of individual tumors on day 22. Data are means±SEM (n=5). The results were compared by one-way ANOVA with Tukey's multiple comparisons test (**P<0.01, *P<0.05). (FIG. 15D) Survival rates of tumor-bearing mice over 70 days (dashed line shows the intersection with median survival for each treatment). The results were compared using the log-rank (Mantel-Cox) test (**P<0.01, *P<0.05). (FIG. 15E) Relative body weight of tumor-bearing mice over 40 days.

FIGS. 16A-16C: Therapeutic effect of CPMV-poly(I:C) particles in the B16F10 melanoma model in vivo. (FIG. 16A) Timeline of B16F10 cell inoculation and i.t. particle injections. (FIG. 16B) Relative tumor volume in tumor-bearing mice following the injection of PBS, CPMV, CPMV+poly(I:C) or CPMV-poly(I:C). Blue arrows indicate the treatment days. Data are means±SEM (n=5). (FIG. 16C) Survival rates of tumor-bearing mice over 60 days (dashed line shows the intersection with median survival for each treatment). The results were compared using the log-rank (Mantel-Cox) test (**P<0.01).

FIGS. 17A-17B: Characterization of native CPMV, CCMV and Qβ particles. (FIG. 17A) Representative TEM image of CPMV, CCMV and Qβ particles. Scale bar=200 nm. (FIG. 17B) SEC profiles of CPMV, CCMV and Qβ particles.

FIGS. 18A-18B: Comparative in vivo therapeutic efficacy of native CPMV, CCMV and Qβ particles in (FIG. 18A) B16F10 and (FIG. 18B) CT26 murine cancer models. Arrows indicate the treatment days.

FIGS. 19A-19C: Characterization of Cy5-labeled CPMV, CCMV and Qβ particles. (FIG. 19A) Schematic diagram showing the synthesis method. (FIG. 19B) Agarose gel electrophoresis. (FIG. 19C) SEC profiles.

FIG. 20: Biodistribution of Cy5-labeled CPMV, CCMV and Qβ particles. Applicant administered Cy5-labeled particles i.d. (100 g/20 L) and monitored fluorescence for 7 days. Yellow dotted lines indicate PBS-treaded control mice.

FIGS. 21A-21F: Characterization of CPMV-poly(I:C) particles. (FIG. 21A) Schematic diagram showing the synthesis method. MeIm—1-methylimidazole; EDC—1-ethyl-3-(3-dimethylaminopropyl)carbodiimide. (FIG. 21B) TEM image of CPMV-poly(I:C) particles. Scale bar=200 nm. (FIG. 21C) Size distribution and (FIG. 21D) surface charge of CPMV and CPMV-poly(I:C) particles dispersed in 0.1 M KP buffer. The boxed inset shows the average diameter (D, nm) and polydispersity (PDI) of the particles. (FIG. 21E) Agarose gel electrophoresis of CPMV, CPMV poly(I:C) and poly(I:C). Left panel shows UV light exposure and right panel shows gel stained with Coomassie Brilliant Blue followed by white light exposure. (FIG. 21F) SEC profile of CPMV poly(I:C). The boxed insets show the elution volume (V, mL) and the absorbance ratio at 260 and 280 nm (A260/280).

FIG. 22: Evaluation of NF-κB/AP-1 activation in RAW-Blue cells by CPMV-poly(I:C). Data are means±SEM (n=6). The results were compared by one-way ANOVA with Tukey's multiple comparisons test (***P<0.001, *P<0.05).

### DETAILED DESCRIPTION

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which can vary by (+) or (−) 15%, 10%, 5%, 3%, 2%, or 1%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 m in diameter and 10 m long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the viral nanoparticle and chemotherapeutic agent of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is the to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

“Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small, secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. Further details as to modern methods of vectors for use in gene transfer can be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

As used herein, “plant viral nanoparticle,” “viral nanoparticle,” “virus like particle” and “bacteriophage VLP” all refer to a viral vector. Non-limiting examples include a nanoparticle derived from a plant virus such as Cowpea chlorotic mottle virus (CCMV), a Cowpea mosaic virus (CPMV), a Physalis mottle virus (PhMV), a Papaya mosaic virus (PapMV), or a Potato virus X (PVX), or a bacteriophage such as Qbeta (Qβ). In one example, the viral vector is comprised of a virus coat protein and a nucleic acid. In another example, the nanoparticle is a plant viral nanoparticle, which is used to delivering a TLR agonist into a mammalian cell or a subject in need thereof.

An “effective amount” or “efficacious amount” refers to the amount of an agent or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

As used herein, a “cancer” is a disease state characterized by the presence in a subject of cells demonstrating abnormal uncontrolled replication and can be used interchangeably with the term “tumor.”

The tumor is not limited and can be any kind of cancer, e.g., solid or blood cancer, e.g., carcinoma or sarcoma. In some embodiments, the cancer is ICI resistant. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, or stomach cancer.

As used herein, the term “hematologic malignancy” refers to cancers with hematopoietic origin. In some instances, the hematologic malignancy is a B-cell malignancy. In some instances, the hematologic malignancy is a lymphoma, optionally a B-cell lymphoma. Exemplary hematologic malignancies include, but are not limited to, Diffuse large B-cell lymphoma (DLBCL), follicular lymphoma, chronic lymphocytic leukemia (CLL), small lymphocytic lymphoma (SLL), mantel cell lymphoma (MCL), marginal zone lymphomas, Burkitt lymphoma, Waldenstram macroglobulinemia, hairy cell leukemia (HCL), primary central nervous system (CNS) lymphoma, or primary intraocular lymphoma.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, 0-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection can be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification marker” or “reporter protein” refer to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the PVX described herein). Homology can be determined by comparing a position in each sequence which can be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide, or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits can be linked by peptide bonds. In another aspect, the subunit can be linked by other bonds, e.g., ester, ether, etc. A protein or peptide must contain at least two amino acids and no limitation is placed on the maximum number of amino acids which can comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation can be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, the term “overexpress” with respect to a cell, a tissue, or an organ expresses a protein to an amount that is greater than the amount that is produced in a control cell, a control issue, or an organ. A protein that is overexpressed can be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer can enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters can be constitutive, inducible, repressible, or tissue-specific, for example. A “promoter” is a control sequence that is a region of a polynucleotide sequence at which initiation and rate of transcription are controlled. It can contain genetic elements at which regulatory proteins and molecules can bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media can be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture can be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium can be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (EVIDM).

“Cryoprotectants” are known in the art and include without limitation, e.g., sucrose, trehalose, and glycerol. A cryoprotectant exhibiting low toxicity in biological systems is generally used.

A non-coding RNA (ncRNA) is an RNA molecule that is not translated into a protein. Non-limiting examples of non-coding RNA include transfer RNAs (tRNAs) and ribosomal RNAs (rRNAs), microRNAs, siRNAs etc.

### Modes for Carrying Out the Disclosure

This disclosure provides compositions and methods of use for viral nanoparticles in combination with a chemotherapeutic agent, wherein the viral nanoparticles comprise, or consist essentially of, or yet further consist of at least one Toll-like Receptor (TLR) agonist conjugated to the nanoparticle. The TLR agonist activates a TLR. In one aspect, the TLRs are TLR3 agonist poly(I:C) and/or TLR7 agonist 1V209. In one embodiment, the chemotherapeutic agent of interest is oxaliplatin. In some aspects, the nanoparticle is derived from a plant virus such as Cowpea chlorotic mottle virus (CCMV), a Cowpea mosaic virus (CPMV), a Physalis mottle virus (PhMV), a Papaya mosaic virus (PapMV), or a Potato virus X (PVX), or a bacteriophage such as Qbeta (Qβ), or any other suitable plant virus or bacteriophage. The nanoparticle can comprise a coat protein (alternatively referred to as capsid protein) from a virus or a combination of viruses. In one embodiment, the nanoparticle is derived from CCMV, CPMV, or Qβ. Methods to prepare the nanoparticles are described herein or described in PCT/US2023/023440 and PCT/US2023/016457. In one embodiment, the plant viral nanoparticle is derived from CCMV, CPMV, or Qβ and comprises, or consists essentially of, or yet further consists of a TLR agonist selected from poly(I:C) and/or TLR7 agonist 1V209. Further provided are the viral nanoparticles conjugated to the TLR agonist and a carrier, such as a pharmaceutically acceptable carrier.

Any bioconjugation or chemical conjugation method would be applicable for the conjugation. Non-limiting examples of chemical conjugation include conjugating a thiol-terminated peptide through a maleimide-PEG-NHS linker targeting lysine groups on the virus or VLP, e.g., CMPV or Qβ coat protein. In some embodiments, a lysine side chain is conjugated to a N-hydroxysuccinimide (NHS) ester and the maleimide of a maleimide-polyethylene glycol8 is conjugated with the c-terminal cysteine of the targeting peptide. Azide/alkyne modified peptides and virus or VLP (CMPV or Qβ coat protein) and click chemistry can also be used for chemical conjugation. For bioconjugation such as genetic fusion, the peptide is added as N-terminal fusion in a CMPV or Qβ coat protein bacteriophage containing the entire nanoparticle. Alternatively, the nanoparticle and S100A9 peptide are recombinantly produced as describe herein.

In some embodiments, the nanoparticle is derived from Cowpea chlorotic mottle virus (CCMV). CCMV is a spherical plant virus that belongs to the Bromovirus genus. Several strains have been identified and include, but not limited to, Carl (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean (S) (Kuhn, 1968. Phytopathology 58:1441-1442), mild (M) (Kuhn, 1979. Phytopathology 69:621-624), Arkansas (A) (Fulton, et al., 1975. Phytopathology 65: 741-742), bean yellow stipple (BYS) (Fulton, et al., 1975. Phytopathology 65: 741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72(9): 768-770).

In some instances, the nanoparticle derived from CCMV comprise, or consists essentially of, or yet further consists of, a plurality of capsid proteins. In some instances, the capsid protein is a wild-type CCMV capsid, optionally expressed by Carl, Car2, type T, soybean (S), mild (M), Arkansas (A), bean yellow stipple (BYS), R, or PSM strain. In other instances, the capsid protein is a modified capsid protein, e.g., comprising, or consisting essentially of, or yet further consisting of, one or more substitutions, insertions, and/or deletions. In some cases, the CCMV capsid comprise, or consists essentially of, or yet further consists of, s the sequence as set forth in the UniProtKB ID P03601:

(SEQ ID NO: 1)
MSTVGTGKLTRAQRRAAARKNKRNTRVVQPVIVEPIASGQGKAIKAWTG
YSVSKWTASCAAAEAKVTSAITISLPNELSSERNKQLKVGRVLLWLGLL
PSVSGTVKSCVTETQTTAAASFQVALAVADNSKDVVAAMYPEAFKGITL
EQLTADLTIYLYSSAALTEGDVIVHLEVEHVRPTFDDSFTPVY,
or
an equivalent thereof.

In some cases, the nanoparticle derived from CCMV is prepared by the method as described in Ali et al., “Rapid and efficient purification of Cowpea chlorotic mottle virus by sucrose cushion ultracentrifugation,” Journal of Virological Methods 141: 84-86 (2007).

In some embodiments, the nanoparticle is derived from Cowpea mosaic virus (CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the nanoparticle derived from CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprises, or consists essentially of, or yet further consists of, the sequence as set forth in the UniProtKB ID P03599 (residues 460-833):

(SEQ ID NO: 2)
MEQNLFALSLDDTSSVRGSLLDTKFAQTRVLLSKAMAGGDVLLDEYLYD
VVNGQDFRATVAFLRTHVITGKIKVTATTNISDNSGCCLMLAINSGVRG
KYSTDVYTICSQDSMTWNPGCKKNFSFTFNPNPCGDSWSAEMISRSRVR
MTVICVSGWTLSPTTDVIAKLDWSIVNEKCEPTIYHLADCQNWLPLNRW
MGKLTFPQGVTSEVRRMPLSIGGGAGATQAFLANMPNSWISMWRYFRGE
LHFEVTKMSSPYIKATVTFLIAFGNLSDAFGFYESFPHRIVQFAEVEEK
CTLVFSQQEFVTAWSTQVNPRTTLEADGCPYLYAIIHDSTTGTISGDFN
LGVKLVGIKDFCGIGSNPGIDGSRLLGAIAQ,
or
an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprises, or consists essentially of, or yet further consists of, s the sequence as set forth in the UniProtKB ID P03599 (residues 834-1022):

(SEQ ID NO: 3)
GPVCAEASDVYSPCMIASTPPAPFSDVTAVTFDLINGKITPVGDDNWNT
HIYNPPIMNVLRTAAWKSGTIHVQLNVRGAGVKRADWDGQVFVYLRQSM
NPESYDARTFVISQPGSAMLNFSFDIIGPNSGFEFAESPWANQTTWYLE
CVATNPRQIQQFEVNMRFDPNFRVAGNILMPPFPLSTETPPL,
or
an equivalent thereof.

In some embodiments, a nanoparticle derived from bacteriophage Qβ comprises, or consists essentially of, or yet further consists of, a plurality of coat proteins. In some embodiments, the coat protein is a wild-type bacteriophage Qβ coat protein. In further embodiments, the coat protein is modified, e.g., comprising, or consisting essentially of, or yet further consisting of, one or more substitutions, insertions, and/or deletions. In some embodiments, a bacteriophage Qβ coat protein comprises, or alternatively consists essentially of, or yet further consists of the sequence as set forth in the UniProtKB ID P03615:

(SEQ ID NO: 4)
MAKLETVTLGNIGKDGKQTLVLNPRGVNPTNGVASLSQAGAVPALEKRV
TVSVSQPSRNRKNYKVQVKIQNPTACTANGSCDPSVTRQAYADVTFSFT
QYSTDEERAFVRTELAALLASPLLIDAIDQLNPAY
or
an equivalent thereof.

Also provided herein are compositions or combinations comprising the viral nanoparticle described herein and a chemotherapeutic agent, such as for example oxaliplatin. The compositions contain the viral nanoparticle and the chemotherapeutic agent in a single composition with a carrier. The combination contains the plant nanoparticle and TLR agonist in a single composition with a carrier, and the chemotherapeutic agent is administered or delivered in a separate composition.

Without wishing to be bound by any particular theory, Applicant hypothesizes that coupling and co-delivery are required to enhance the anti-tumor efficacy of TLR agonists. For example, the delivery of 1V209 or poly(I:C) conjugated to viral nanoparticles can enhance their efficacy due to the multivalent presentation, prolonging of tumor residence time, and targeting of innate immune cells mediated by the viral nanoparticle.

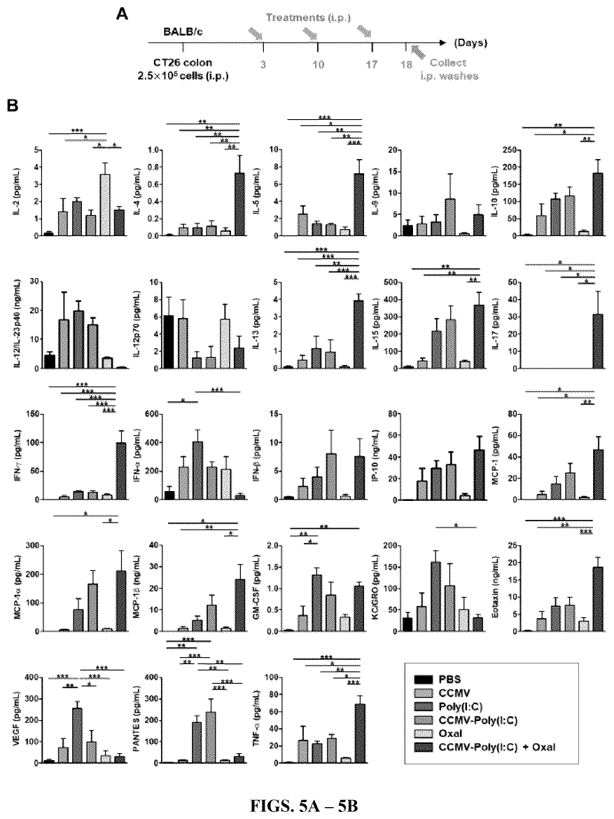

In one example, Applicant has demonstrated a synergistic effect when TLR agonists and chemotherapeutic agents are added in combination to a cell or subject in need. in particular, the combination of CCMV-Poly(I:C)+oxaliplatin was more effective than CCMV, CCMV-Poly(I:C), or oxaliplatin, for example in tumor tissue penetration (FIG. 7) and induction of expression of select chemokines and cytokines (FIG. 5).

### Composition of a TLR-Agonist and a Chemotherapeutic Agent

In some embodiments, this disclosure provides a nanoparticle containing a TLR agonist that activates a TLR, and a chemotherapeutic agent. In the embodiments described herein, the TLR agonists are conjugated to virus coat proteins to form the nanoparticle. In some embodiments, this disclosure provides a composition comprised of a VLP containing a TLR agonist and a chemotherapeutic agent. In some embodiments the composition comprises, or alternatively consists essentially of, or yet further consists of at least one viral nanoparticle comprised of a toll-like receptor (TLR) agonist selected from a double-stranded RNA (dsRNA) or a synthetic dsRNA that activates a TLR conjugated to a virus coat protein, and at least one chemotherapeutic agent. In some embodiments, the composition comprises a viral nanoparticle comprising at least two TLR agonists and at least one chemotherapeutic agent. In compositions with viral nanoparticles comprising at least two TLR agonists, the TLR agonists can activate the same or different TLRS. In some embodiments there are two or more viral nanoparticles in the composition, wherein the viral nanoparticles comprise TLR agonists which activates the same or different TLR. In some embodiments there are two or more chemotherapeutic agents in the composition. The TLR can be selected from TLR1, TLR2, TLR3, TLR4, TLR5, TLR6, TLR7, TLR8, or TLR9, and in one aspect is TLR3. In some embodiments the virus is a plant virus selected from CPMV or CMMV, or other suitable plant viruses. In some embodiments the virus is a bacteriophage selected from Qβ, or other suitable bacteriophages. The chemotherapeutic agent can be selected from 5-fluorouracil, methyl-CCNU, oxaliplatin, irinotecan, mitomycin, cytarabine, doxorubicin, or cyclophosphamide, and is preferably oxaliplatin. In compositions with at least two TLR agonists, in one aspect, the two TLR agonists are 1V209 and poly(I:C) and the viral nanoparticle is derived from CPMV, CMMV, or Qβ.

According to one embodiment, the TLR is TLR3, the TLR3 agonist is poly(I:C), and the chemotherapeutic agent comprises or consists essentially of oxaliplatin. According to another embodiment, the TLR is TLR7, the TLR7 agonist is 2-methoxyethoxy-8-oxo-9-(4-carboxybenzyl)adenine (1V209), and the chemotherapeutic agent comprises or consists essentially of oxaliplatin and the viral nanoparticle is derived from CPMV, CMMV, or Qβ.

Methods to prepare the viral nanoparticles and compositions are described herein or described in PCT/US2023/023440 and PCT/US2023/016457.

In some aspects, the TLR agonists and chemotherapeutic agents are commercially available.

Further provided are compositions comprising: the viral nanoparticles comprised of the virus derived coat protein conjugated to the TLR agonist, the chemotherapeutic agent and a carrier, such as a pharmaceutically acceptable carrier.

As described herein, the viral nanoparticles as disclosed or described herein can also include a carrier and/or a detectable label. The viral nanoparticle and chemotherapeutic agents of the present disclosure are combined with different carriers. Thus, this disclosure also provides compositions containing the viral nanoparticle, chemotherapeutic agent, and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding viral nanoparticle filaments, or will be able to ascertain such, using routine experimentation.

In another aspect, the composition further comprises an additional therapeutic agent. In some embodiments the additional therapeutic agent is an anti-cancer therapy. In some embodiments the additional therapeutic agent comprises an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the additional therapeutic agent comprises an inhibitor of the enzyme poly ADP ribose polymerase (PARP). Exemplary PARP inhibitors include, but are not limited to, olaparib (AZD-2281, Lynparza®, from Astra Zeneca), rucaparib (PF-01367338, Rubraca®, from Clovis Oncology), niraparib (MK-4827, Zejula®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprises an immune checkpoint inhibitor. Exemplary checkpoint inhibitors include:
PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736;
PD-L2 inhibitors such as GlaxoSmithKline's AMP-224 (Amplimmune), and rHIgM12B7;
PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANBO11, antibody MDX-1 106 (ONO-4538), Bristol-Myers Squibb's human IgG4 monoclonal antibody nivolumab (Opdivo®, BMS-936558, MDX1106), AstraZeneca's AMP-514 and AMP-224, and Pidilizumab (CT-011) from CureTech Ltd;
CTLA-4 inhibitors such as Bristol Meyers Squibb's anti-CTLA-4 antibody ipilimumab (also known as Yervoy®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam;
LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;
B7-H3 inhibitors such as MGA271;
KIR inhibitors such as Lirilumab (IPH2101);
CD137 inhibitors such as urelumab (BMS-663513, Bristol-Myers Squibb), PF-05082566 (anti-4-1BB, PF-2566, Pfizer), or XmAb-5592 (Xencor);
PS inhibitors such as Bavituximab;
and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprises pembrolizumab, nivolumab, tremelimumab, or ipilimumab.

In some cases, the additional therapeutic agent comprises an antibody such as alemtuzumab, trastuzumab, ibritumomab tiuxetan, brentuximab vedotin, ado-trastuzumab emtansine, or blinatumomab.

In some cases, the additional therapeutic agent comprises a cytokine. Exemplary cytokines include, but are not limited to, IL-Iβ, IL-6, IL-7, IL-10, IL-12, IL-15, IL-21, or TNFα.

In some cases, the additional therapeutic agent comprises an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprises use of allogeneic T lymphocytes with, e.g., anti-tumor activity, expansion of the T lymphocytes in vitro, and subsequent infusion of the expanded allogeneic T lymphocytes into a subject in need thereof.

Pharmaceutical compositions of the present disclosure can be administered in a manner appropriate to the disease to be treated or prevented. The quantity and frequency of administration will be determined by such factors as the condition of the patient, and the type and severity of the patient's disease, although appropriate dosages can be determined by clinical trials.

In some embodiments, the pharmaceutical composition and formulations described herein are administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intratechal administration. In some instances, the pharmaceutical composition is formulated for local administration. In other instances, the pharmaceutical composition is formulated for systemic administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkinsl999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet©, boric acid, sodium benzoate, sodium acetate, sodium chloride, leucine, a polyethylene glycol (e.g., PEG-4000) or a methoxypolyethylene glycol such as Carbowax™ sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Methods Using Compositions Comprising at Least One TLR Agonist and Chemotherapeutic Agent

The compositions of the present disclosure can be used to deliver a TLR agonist and a chemotherapeutic agent to a mammalian cell. In some embodiments the TLR agonist and chemotherapeutic agent are delivered by contacting the cell with the composition in vitro. In some embodiments the TLR agonist and chemotherapeutic agent are delivered by contacting the cell with the composition in vivo. Contacting the mammalian cell in vitro or in vivo can prevent cell growth and in one aspect, the cell is a cancer cell. Non-limiting examples of cancer cells include carcinoma cells, sarcoma cells, or blood cancer cells. Additionally, the cancer cells can be B16F10 or CT26 cells. The cells can be primary cancer cells (such as those isolated from a tissue biopsy) or a cultured cell that can be commercially available from American Type Culture Collection (ATCC). In other aspects, the cell is a RAW-Blue cell.

Further, this disclosure also provides methods to activate the immune response of a cell by contacting the cell with the composition in vitro. In some embodiments the TLR agonist and chemotherapeutic agent are delivered by contacting the cell with the composition in vivo. Contacting the mammalian cell in vitro or in vivo can agent can stimulate the immune response of the cell to clear disease and/or restore cell function.

The compositions of the present disclosure can be used to treat to treat, inhibit, delay, slow down, or prevent relapse of cancer in a subject by administering to the subject a composition as described herein. The compositions can be administered either alone or in combination with diluents, known anti-cancer therapeutics, and/or with other components such as cytokines or other cell populations that are immunostimulatory. They can be administered as a first line therapy, a second line therapy, a third line therapy, or further therapy. The disclosed nanoparticles and/or composition can be combined with other therapies (e.g., radiation, surgery etc.). Appropriate treatment regimens will be determined by the treating physician or veterinarian. In one embodiment, disclosed herein is a method of inhibiting the growth of a tumor and/or treating a cancer and/or preventing relapse of cancer in a subject in need thereof, comprising, or alternatively consisting essentially of, or yet further consisting of administering to the subject an effective amount of the compositions provided herein. The cancer can be localized or metastatic.

In one embodiment, the tumor is a solid tumor. The solid tumor could be a melanoma, a colon carcinoma, a breast carcinoma and/or a brain tumor. In one aspect, the cancer to be treated is a carcinoma, sarcoma, or blood cancer, a colon cancer, a rectal cancer, or a melanoma, and further optionally a Stage I, Stage II, a Stage III or a Stage IV cancer.

The methods are useful to treat subjects such as humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In certain embodiments the subject has or is suspected of having a neoplastic disorder, neoplasia, tumor, malignancy or cancer.

For the above methods, an effective amount is administered, and administration of the cell or population serves to attenuate any symptom or prevent additional symptoms from arising. When administration is for the purposes of preventing or reducing the likelihood of cancer recurrence or metastasis, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal. Preferably the composition is administered intraperitoneally or subcutaneously.

The methods provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies e.g., surgical recession, chemotherapy, radiation. In one aspect, treatment excludes prophylaxis.

Further, this disclosure provides a method for triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells in a subject in need by administering to the subject an effective amount of a composition described herein. The immunostimulatory cytokine or chemokine can be selected from IL-4, IL-5, IL-13, IL-15, IFN-γ, IP-10, MCP-1, MCP-1α, MCP-1β, Eotoxin, or TNF-α.

### Kits Using Compositions Comprising at Least One TLR Agonist and Chemotherapeutic Agent

In some embodiments, one or more of the compositions described or disclosed herein are contained in a kit. Accordingly, in some embodiments, provided herein is a kit comprising, consisting essentially of, or consisting of one or more compositions disclosed herein and instructions for their use.

### Methods Using a Combination TLR-Agonist and a Chemotherapeutic Agent Treatment

The following methods describe a combination treatment of a viral nanoparticle comprising dsRNA or synthetic dsRNA capable of activating a TLR (TLR agonist) and a chemotherapeutic agent.

Methods to prepare the viral nanoparticles are described herein or described in PCT/US2023/023440 and PCT/US2023/016457.

In some embodiments, this disclosure provides a nanoparticle containing a TLR agonist that activates a TLR, and a chemotherapeutic agent. In the embodiments described herein, the TLR agonists are conjugated to virus coat proteins to form the nanoparticle. The TLR can be selected from TLR1, TLR2, TLR3, TLR4, TLR5, TLR6, TLR7, TLR8, or TLR9, and in one aspect is TLR3. In some embodiments the virus is a plant virus selected from CPMV or CMMV, or other suitable plant viruses. In some embodiments the virus is a bacteriophage selected from Qβ, or other suitable bacteriophages. The chemotherapeutic agent can be selected from 5-fluorouracil, methyl-CCNU, oxaliplatin, irinotecan, mitomycin, cytarabine, doxorubicin, or cyclophosphamide, and is preferably oxaliplatin. According to one embodiment, the TLR is TLR3, the TLR3 agonist is poly(I:C), and the chemotherapeutic agent comprises or consists essentially of oxaliplatin. In another embodiment, the TLR is TLR3, the TLR3 agonist is poly(I:C), and the chemotherapeutic agent is a different suitable chemotherapeutic agent.

This disclosure provides a method to prevent mammalian cell growth by contacting the cell with a viral nanoparticle which comprises dsRNA or synthetic dsRNA capable of activating a TLR (TLR agonist), and a chemotherapeutic agent. In some embodiments the contacting the cell is done in vitro. In some embodiments the contacting the cell is done in vivo. Contacting the mammalian cell in vitro or in vivo can prevent cell growth and in one aspect, the cell is a cancer cell. Non-limiting examples of cancer cells include carcinoma cells, sarcoma cells, or blood cancer cells. Additionally, the cancer cells can be B16F10 or CT26 cells. The cells can be primary cancer cells (such as those isolated from a tissue biopsy) or a cultured cell that can be commercially available from American Type Culture Collection (ATCC). In other aspects, the cell is a RAW-Blue cell.

In some embodiments the viral nanoparticle and chemotherapeutic agent are applied simultaneously. In other embodiments the viral nanoparticle and chemotherapeutic agent are applied sequentially. The viral nanoparticle and chemotherapeutic agent can be administered in one dose or at least two separate doses.

Further, this disclosure also provides methods to activate the immune response of a cell by contacting the cell with a viral nanoparticle which comprises dsRNA or synthetic dsRNA capable of activating a TLR (TLR agonist), and a chemotherapeutic agent. The combination of the viral nanoparticle and chemotherapeutic agent can stimulate the immune response of the cell to clear disease and/or restore cell function. In some embodiments the contacting the cell is done in vitro.

In some embodiments the contacting the cell is done in vivo. In some embodiments the viral nanoparticle and chemotherapeutic agent are applied simultaneously. In other embodiments the viral nanoparticle and chemotherapeutic agent are applied sequentially. The viral nanoparticle and chemotherapeutic agent can be administered in one dose or at least two separate doses.

This disclosure also provides methods to treat, inhibit, delay, slow down, or prevent relapse of cancer in a subject in need by administering to the subject an effective amount of a viral nanoparticle which comprises dsRNA or synthetic dsRNA capable of activating a TLR (TLR agonist), and a chemotherapeutic agent. In some embodiments the viral nanoparticle and chemotherapeutic agent are applied simultaneously. In other embodiments the viral nanoparticle and chemotherapeutic agent are applied sequentially. The viral nanoparticle and chemotherapeutic agent can be administered in one dose or at least two separate doses.

Further, this disclosure provides a method for triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells in a subject in need by administering to the subject an effective amount of a viral nanoparticle which comprises dsRNA or synthetic dsRNA capable of activating a TLR (TLR agonist), and a chemotherapeutic agent. In some embodiments the viral nanoparticle and chemotherapeutic agent are applied simultaneously. In some embodiments the chemotherapeutic agent is packaged in the viral nanoparticle. In other embodiments the chemotherapeutic agent and viral nanoparticle are co-administered. In some embodiments the nanoparticle and chemotherapeutic agent are applied sequentially. The viral nanoparticle and chemotherapeutic agent can be administered in one dose or at least two separate doses. The immunostimulatory cytokine or chemokine can be selected from IL-4, IL-5, IL-13, IL-15, IFN-γ, IP-10, MCP-1, MCP-1ca, MCP-1β, Eotoxin, or TNF-α.

In some aspects, the plant virus nanoparticle is derived from a plant virus such as Cowpea chlorotic mottle virus (CCMV), a Cowpea mosaic virus (CPMV), a Physalis mottle virus (PhMV), a Papaya mosaic virus (PapMV), or a Potato virus X (PVX), or a bacteriophage such as Qbeta (Qβ), or other suitable plant virus or bacteriophage. The nanoparticle can comprise a capsid protein, preferably from CCMV, CPMV, or a combination of the two viruses. Preferably, the TLR is TLR3. In some embodiments the TLR3 agonist is poly(I:C). In some aspects, the chemotherapeutic agent is selected from 5-fluorouracil, methyl-CCNU, oxaliplatin, irinotecan, mitomycin, cytarabine, doxorubicin, or cyclophosphamide. Preferably, the chemotherapeutic agent is oxaliplatin. Preferably the methods comprise administering a viral nanoparticle comprising the TLR3 agonist poly(I:C) and the chemotherapeutic agent oxaliplatin.

The viral nanoparticle comprising at TLR agonist and chemotherapeutic agent can be combined with other therapies (e.g. radiation, surgery, etc.). Appropriate treatment regimens will be determined by the treating physician or veterinarian. In one embodiment, disclosed herein is a method of inhibiting the growth of a tumor and/or treating a cancer and/or preventing relapse of cancer in a subject in need thereof, comprising, or alternatively consisting essentially of, or yet further consisting of administering to the subject an effective amount of the viral nanoparticle and chemotherapeutic agent disclosed herein.

In one embodiment, the tumor is a solid tumor. The solid tumor could be a melanoma, a colon carcinoma, a breast carcinoma and/or a brain tumor, e.g., a glioblastoma. In one aspect, the cancer to be treated is a carcinoma, sarcoma, or blood cancer, a colon cancer, a rectal cancer, or a melanoma, and further optionally a Stage I, Stage II, a Stage III or a Stage IV cancer.

The methods are useful to treat subjects such as humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In certain embodiments the subject has or is suspected of having a neoplastic disorder, neoplasia, tumor, malignancy or cancer.

For the above methods, an effective amount of the composition is administered, and administration of the cell or population serves to attenuate any symptom or prevent additional symptoms from arising. When administration is for the purposes of preventing or reducing the likelihood of cancer recurrence or metastasis, the compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal. Preferably the nanoparticle is administered intraperitoneally or subcutaneously.

The methods provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies e.g., surgical recession, chemotherapy, radiation. In one aspect, treatment excludes prophylaxis.

### Examples

### Example 1: Enhanced Efficacy of a TLR3 Agonist Delivered by Cowpea Chlorotic Mottle Virus Nanoparticles

Intratumoral immunotherapies are those that are administered directly into a tumor to remodel the local tumor microenvironment and stimulate systemic anti-tumor immunity. Various small molecule Toll-like receptor (TLR) agonists undergoing development as intratumoral immunotherapies, and in this work TLR3 agonist poly(I:C). Because small molecule therapeutics often suffer rapid washout effects and ineffective immune cell uptake, Applicant encapsulated poly(I:C) into nanoparticles derived from cowpea chlorotic mottle virus (CCMV). CCMV-poly(I:C) particles were prepared by controlled disassembly and reassembly and were found to be stable and immunogenic in vitro and in vivo. The particles (but not the separate components) stimulated the activity of macrophages in vitro and were able to reduce tumor growth and prolong survival in mouse models of colon cancer and melanoma. Applicant also combined the CCMV-poly(I:C) formulation with the antineoplastic drug oxaliplatin and found the combination therapy to be even more potent, strongly inhibiting tumor growth and increasing the survival of the tumor-bearing mice, with up to 75-100% of the mice surviving after 40 days. The analysis of immune markers revealed that CCMV-poly(I:C) VLPs with oxaliplatin promoted the infiltration and activation of CD4+ and CD8+ cells and the production of IL-4 and IFN-7, indicating a synergistic immunogenic effect. The combined treatment also enhanced the rate of apoptosis and immunogenic cell death (ICD). Applicant's data support the development of combination therapies involving plant viruses and antineoplastic drugs to attack tumors directly and via the activation of innate and adaptive immune responses.

Here Applicant focused on polyinosinic:polycytidylic acid (poly(I:C)), which resembles the structure of dsRNA and agonizes TLR3. TLR3 recognizes double stranded RNA to activate innate immune cells (macrophages, dendritic cells) to become APCs leading to CD4⁺ T cell responses to switch from Th2 to Th1 while boosting the CD8⁺ T cell response and inhibiting T_{reg} cells [10]. TLRs are activated by a variety of synthetic agonists, including polyinosinic:polycytidylic acid (poly(I:C)), which resembles the structure of dsRNA and interacts with TLR3. The intratumoral application of free poly(I:C) is hampered by rapid washout effects and limited cell uptake—to overcome these shortcomings and control tissue diffusivity and enhance cell uptake, Applicant opted to encapsulate poly(I:C) into nanoparticles derived from cowpea chlorotic mottle virus (CCMV).

Applicant developed CCMV particles loaded with the TLR3 agonist poly(I:C) and combined it with oxaliplatin, and a chemotherapeutic [18]. Without wishing to be bound by theory, the underlying hypothesis is that the chemotherapy induces cancer cell death to release tumor associated and neoantigens to be processed by innate immune cells recruited and activated by the TLR3 agonist, therefore resulting in improved therapy success. Applicant tested this hypothesis in a mouse model of colon cancer and investigated the underlying immunological mechanism through a combination of chemo/cytokine analysis, immunological cell profiling, and tumor histology imaging.

### Materials and Methods

### Preparation of CCMV-Poly(I:C) VLPs

CCMV was produced as previously described [19]. Briefly, black-eyed pea No. 5 plants were mechanically infected with CCMV and leaves were harvested 14 days post-inoculation. The leaves were homogenized, extracted with chloroform, and CCMV was purified by ultracentrifugation on a sucrose cushion. CCMV capsid proteins were acquired by overnight dialysis in disassembly buffer (0.05 M Tris-HCl, 0.5 M CaCl₂, 0.001 M EDTA, 0.001 M DTT, 0.0005 M PMSF, pH 7.5) and the precipitated RNA was removed by centrifugation (15,000 g, 2 h, 4° C.). The purity of the capsid proteins was determined by measuring the UV/vis absorbance ratio (A280/260˜1.5) before resuspending in protein buffer (0.02 M Tris-HCl, 1 M NaCl, 0.001 M EDTA, 0.001 M DTT, 0.001 M PMSF, pH 7.2). To prepare the CCMV-poly(I:C) VLPs, the capsid proteins were reassembled with 0.2-1 kb poly(I:C) (InvivoGen) at a 6:1 mass ratio in reassembly buffer (0.5 M Tris-HCl, 0.5 M NaCl, 0.1 M KCl, 0.05 M MgCl₂, 0.01 M DTT, pH 7.2). The reconstituted CCMV-poly(I:C) particles were then stored in virus suspension buffer (0.5 M sodium acetate, 0.08 M magnesium acetate, pH 4.5) at 4° C. The particle concentration was determined using a Pierce BCA protein kit (Thermo Fisher Scientific). The encapsulation of poly(I:C) was confirmed using a Quant-it RiboGreen RNA assay kit (Thermo Fisher Scientific).

### Analysis of CCMV-Poly(I:C) VLPs

The UV/vis absorbance profiles of CCMV and CCMV-poly(I:C) were acquired using a NanoDrop spectrophotometer (Thermo Fisher Scientific). The concentration of CCMV was calculated according to Beer's Law (A=εbc), where A is the absorbance, c is the molar absorptivity coefficient of 5.85 mg⁻¹ cm⁻¹, b is the length of the light path, and c is the concentration. The CCMV and CCMV-poly(I:C) particles were loaded onto 1.2% (w/v) agarose native gels (10 g per lane) stained with GelRed for nucleic acids and separated by electrophoresis in virus separation buffer (0.1 M sodium acetate, 1 mM EDTA, pH 5.5) for 1 h at 60 V and 4° C. [19]. The gels were imaged using an AlphaImager System under UV light to detect RNA before staining with Coomassie Brilliant Blue to detect protein under white light. For transmission electron microscopy (TEM), CCMV and CCMV-poly(I:C) were dispersed in deionized water at 100 g/mL and dropped onto Formvar carbon film-coated copper grids (Ted Pella) and incubated for 2 min, followed by washing twice with deionized water for 1 min. The grids were coated with 2% (w/v) uranyl acetate for 2 min and images were acquired using a JEOL JEM-1400Plus microscope. The particle size was measured using ImageJ. CCMV and CCMV-poly(I:C) size and integrity was further corroborated using fast protein liquid chromatography (FPLC) on the ÄKTA Pure™ chromatography system (Cytiva). Particles were analyzed using a Superose 6 Increase column at a flow rate of 0.5 mL/min; RNA was detected at 260 nm and protein at 280 nm.

### Cell Cultures

RAW-Blue cells (InvivoGen) were grown in Dulbecco's Modified Eagle's Medium (DMEM) supplemented with 10% (v/v) heat-sterilized fetal bovine serum (FBS; Atlanta Biologicals) and 1% (v/v) penicillin/streptomycin (p/s; Thermo Fisher Scientific). Normocin (50 mg/mL) and zeocin (100 mg/mL) were also added according to the supplier's instructions (InvivoGen). CT26 cells obtained from the ATCC were grown in Roswell Park Memorial Institute (RPMI) 1640 medium and DMEM, respectively, each supplemented with 10% (v/v) FBS and 1% (v/v) p/s.

### In Vitro Immunogenicity

RAW-Blue cells (1×10⁵ cells/well) were seeded in 96-well plates and incubated with CCMV (1 g), poly(I:C) (0.3 g) or CCMV-poly(I:C) (1 g) for 24 h. Lipopolysaccharide (LPS; InvivoGen) was used as a positive control. After the incubation, 20 μL of the supernatant was mixed with 180 μL of Quanti-Blue solution (InvivoGen) and incubated at room temperature for 6 h. The absorbance was then measured at 630 nm using a microplate reader (Tecan).

### In Vivo Anti-Tumor Efficacy

Female BALB/c and C57BL/6 mice (6-7 weeks old) were purchased from the Jackson Laboratory. All animal studies were approved by and conducted according to the regulation of the Institutional Animal Care and Use Committee of the University of California, San Diego. CT26 cells were administered via the intraperitoneal (i.p.) and subcutaneous (s.c.) routes. To establish the CT26 colon cancer i.p. model, BALB/c mice were injected i.p. with 2.5×10⁵ CT26 cells/200 μL PBS followed by i.p. treatment with CCMV (200 g in 200 μL PBS), poly(I:C) (˜60 g in 200 μL PBS), CCMV-poly(I:C) (200 g in 200 μL PBS), oxPt (5 mg/kg) or CCMV-poly(I:C)+oxPt (doses as above) 3 days after cell inoculation. Treatments were administered three times at weekly intervals. The body circumference and weight were recorded for 40 days at 2-day intervals and mice were euthanized when the circumference reached ˜8 cm. To establish the CT26 colon cancer s.c. model, CT26 cells (1.5×10⁵ cells in 15 μL PBS) were mixed with 15 μL Matrigel (Corning) and injected s.c. into the left flank of BALB/c mice. When tumors grew to ˜30 mm³, mice were treated intratumorally (i.t.) with CCMV (200 g in 30 L). The tumor volumes, calculated using the equation tumor width²×tumor length)/2, and body weight were recorded for 40 days at 2-day intervals and mice were euthanized when the tumor volume reached ˜1500 mm³.

### Mesoscale Discovery (MSD) Assay

BALB/c mice were challenged i.p. with 1.5×10⁵ CT26 cells in 200 μL PBS and treated i.p. from 3 days post inoculation three times at weekly intervals with CCMV (200 g in 200 μL PBS), poly(I:C) (˜60 g in 200 μL PBS), CCMV-poly(I:C) (200 g in 200 μL PBS), oxPt (5 mg/kg) or CCMV-poly(I:C)+oxPt (doses as above). Applicant collected i.p. washes on day 24 and determined the level of cytokines and chemokines using an MSD assay using a customizable U-PLEX analyzing the following cytokines/chemokines according to manufacturer's instructions: GM-CSF, IFN-α, IFN-β, IFN-γ, IL-2, IL-4, IL-5, IL-9, IL-10, IL-12p70, IL-12/IL-23p40, IL-13, IL-15, IL-17A, KC/GRO, eotaxin, IP-10, MCP-1, MIP-la, MIP-1(3, TNF-α, VEGF-A, and RANTES. The plate was read using a MESO QuickPlex SQ 120 instrument and analyzed using MSD Workbench 4.0 software.

### Flow Cytometry

The i.p. washes of CT26 tumor-bearing mice were stained with Live/Dead aqua (1:1000; Thermo Fisher Scientific) and blocked with CD16/CD32 (1:1000; BioLegend) before staining for CD45 (pacific blue, 30-F11), CD3F (APC/Cyanine 7, 145-2C11), CD8a (APC, 53-6.7), and CD4 (FITC, GK1.5) (1:1000; BioLegend). The stained cells were analyzed using a BD FACSCelesta and a minimum of 10,000 cells was acquired. Data were analyzed using FlowJo software.

### Enzyme-Linked Immunosorbent Spot (ELISPOT) Assay

Spleens were harvested from CT26 tumor-bearing mice on day 24 and processed using a spleen dissociation kit and gentleMACS dissociator (Miltenyi Biotec). Splenocytes (1×10⁵ cells) were seeded in 96-well plates and co-incubated with CTL-Test medium (negative control), CT26 (1×10⁵ cells), 4T1 (1×10⁵ cells), and phorbol myristate acetate/ionomycin (PMA/Iono) (positive control), respectively. The level of IL-4 and IFN-γ was measured using a Mouse IFN-γ/IL-4 Double-Color ELISPOT kit (ImmunoSpot) according to the manufacturer's instructions.

### Histological Evaluation

When CT26 s.c. tumors had reached a volume of ˜30 mm³, mice were challenged i.t. three times with VLPs at weekly intervals. Tumors were harvested on day 24 and fixed with 4% formaldehyde (Thermo Fisher Scientific) before embedding in OCT compound. Applicant then prepared 8-μm sections and stained them with the terminal deoxynucleotidyltransferase dUTP nick end-labeling (TUNEL) kit (Promega) and anti-calreticulin (anti-CRT) antibodies (Invitrogen, diluted 1:500) to observe apoptosis and immunogenic cell death (ICD), respectively. Nuclei were visualized with DAPI. Fluorescent images were acquired by a confocal microscopy and fluorescence intensities were quantified using ImageJ.

### Statistical Analysis

Data were analyzed in GraphPad Prism v8 and are presented as means±standard deviations. For multiple comparisons, statistical significance (p<0.05) was determined by one-way analysis of variance (ANOVA) with a post hoc Tukey's honest significant difference (HSD) test.

### Results and Discussion

### Preparation and Characterization of CCMV-Poly(I:C) VLPs

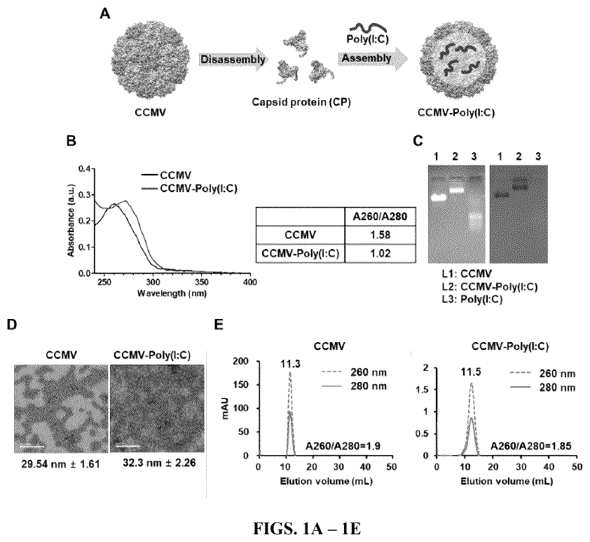

CCMV-poly(I:C) particles were prepared by the disassembly of native CCMV into capsid proteins and viral RNA, followed by the precipitation of the RNA [20]. The capsid proteins and poly(I:C) were then mixed at a mass ratio of 6:1 and assembled in an acidic buffer [21] (FIG. 1A). Native agarose gel electrophoresis indicated assembly CCMV-poly(I:C) particles with colocalization of RNA (GelRed) and protein bands (Coomassie) and the absence of free poly(I:C) (FIG. 1B). The size and morphology of the particles were determined by TEM. The diameter of the CCMV-poly(I:C) particles was 32.3±2.26 nm, slightly larger than native CCMV at 29.54±1.61 nm (FIG. 1C). The purity and integrity of CCMV-poly(I:C) were also confirmed by the unique SEC peak at an elution volume of ˜11 mL for both particles (FIG. 1D). The A260/280 ratios of the CCMV-poly(I:C) and CCMV peaks were ˜1.85 and ˜1.9, which is consistent with intact CCMV.

### In Vitro Immunogenicity

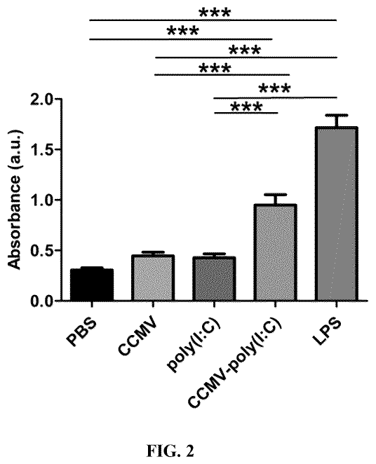

The immunogenicity of CCMV-poly(I:C) VLPs was initially tested in vitro using RAW-Blue cells and a Quanti-Blue assay. SEAP levels in cells treated with CCMV and free poly(I:C) did not differ significantly from the PBS-treated control, indicating no immunomodulatory effects of CCMV alone, which is consistent with our previous observations [22]. However, CCMV-poly(I:C) VLPs increased the SEAP levels by 3.1-fold compared to PBS, 2.3-fold compared to CCMV, and 2.2-fold compared to free poly(I:C) (FIG. 2). This result confirmed the in vitro immunogenicity of CCMV-poly(I:C) and suggested that the formulation is also likely to show immunogenicity in vivo be activating the TLR3 signaling pathway. Similarly, in our previous work CCMV-ODN1826 particles were shown to activate RAW-Blue cells although in this case the pathway involved was TLR9 [17].

Enhanced Therapeutic Efficacy of CCMV-Poly(I:C) in Combination with Oxaliplatin

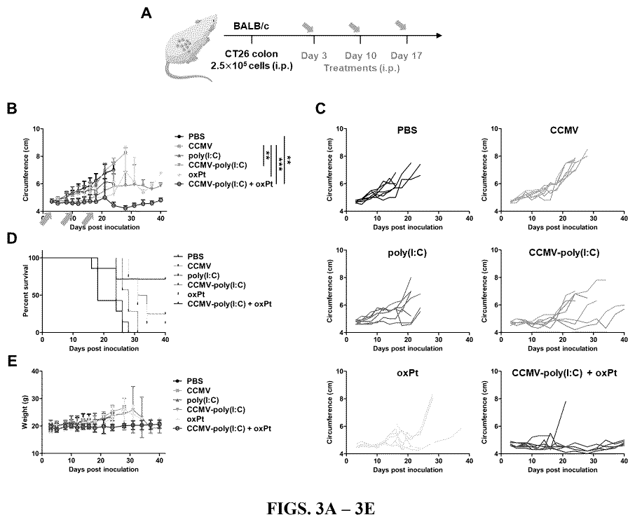

To investigate the therapeutic efficacy of CCMV-poly(I:C) alone and combined with oxaliplatin, Applicant assessed the effect of each treatment in vivo using the CT26 model of colon cancer. Mice with tumors induced by i.p. inoculation with CT26 cells were assigned to six treatment groups as follows: PBS (negative control), CCMV, poly(I:C), CCMV-poly(I:C), oxaliplatin (oxPt), or CCMV-poly(I:C)+oxPt. CCMV alone did not inhibit tumor growth compared to the PBS control, whereas free poly(I:C) had a slight inhibitory effect, albeit not statistically significant (FIG. 3A). In contrast, CCMV-poly(I:C) VLPs significantly inhibited tumor growth and also slightly increased the survival rate compared to mice treated with CCMV or poly(I:C) alone, as did the free oxaliplatin treatment. Similarly, separate treatments with CCMV and ODN1826 had a minimal impact on CT26 tumors but the CCMV-ODC1826 encapsulated VLPs had a significant effect [17]. However, the most potent outcome was achieved by the combination of CCMV-poly(I:C) and oxaliplatin, which not only significantly inhibited tumor growth but also had a remarkable effect on survival, with 75% of the mice in this group surviving after 40 days (FIGS. 3B-3D). The body weight of CT26-bearing mice treated with PBS, CCMV or poly(I:C) increased due to the growth of the tumor, whereas there was no significant gain or loss of body weight in the CCMV-poly(I:C)+oxPt group (FIG. 3E). This is reminiscent of the effect achieved by combining PVX nanoparticles with doxorubicin, which enhanced the efficacy compared to the individual treatments in a B16F10 melanoma model but only when the VLP and drug were mixed, not when they were physically joined by conjugation, indicating the two components act synergistically against different targets [13].

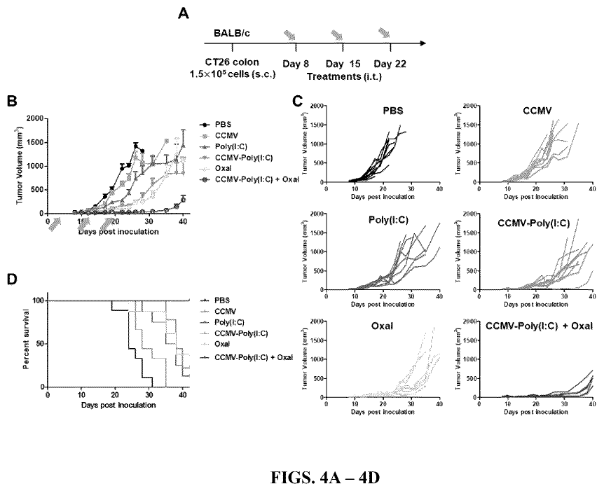

The results observed in the CT26 i.p. model were replicated in the CT26 s.c. model (FIG. 4A). In the PBS and CCMV groups, the tumors grew 47.5-fold and 52.4-fold, respectively, by day 26. In the poly(I:C) group, tumor growth was suppressed but not to a statistically significantly extent (30-fold growth by day 26). In the CCMV-poly(I:C) and oxPt groups, the tumors grew to similar volumes of ˜242 and ˜233 mm³, respectively. However, in the CCMV-poly(I:C)+oxPt group the volume of the tumors on day 26 was just 30 mm³ (FIG. 4B, FIG. 4C). The CCMV-poly(I:C)+oxPt treatment also had the most significant effect on survival, with 100% of the mice in this group remaining alive after 40 days (FIG. 4D).

Enhanced Immunogenicity of CCMV-Poly(I:C) in Combination with Oxaliplatin

The underlying mechanisms of the observed in vivo responses were determined by the analysis of i.p. washes and splenocytes. Applicant used an MSD assay to measure the levels of cytokines and chemokines in i.p. washes collected from immunized mice (FIG. 5). As shown in FIG. 5B, PBS or CCMV-treated tumors did not show any difference in the production of cytokines or chemokines in i.p. washes. However, CCMV-poly(I:C) or CCMV-poly(I:C)+oxPt showed considerable difference in the level of cytokines or chemokines compared to CCMV and poly(I:C) alone. Interestingly, CCMV-poly(I:C)+oxPt notably increased the some cytokines and chemokines, including IL-4, IL-5, IL-13, IL-15, IFN-γ, IP-10, MCP-1, MCP-1α, MCP-1, Eotoxin, and TNF-α.

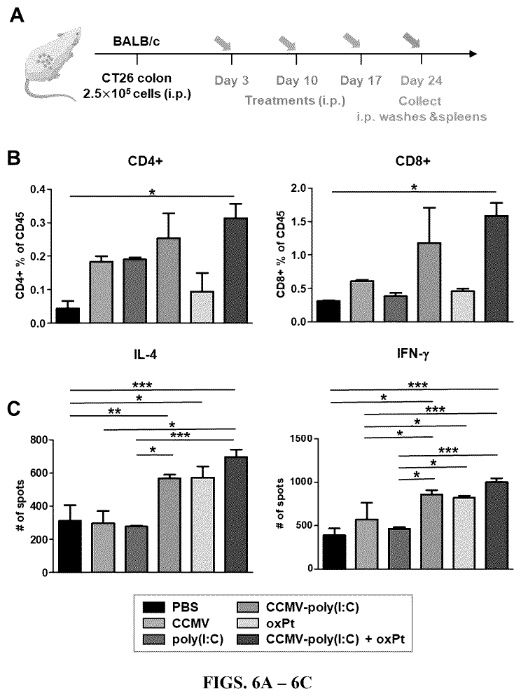

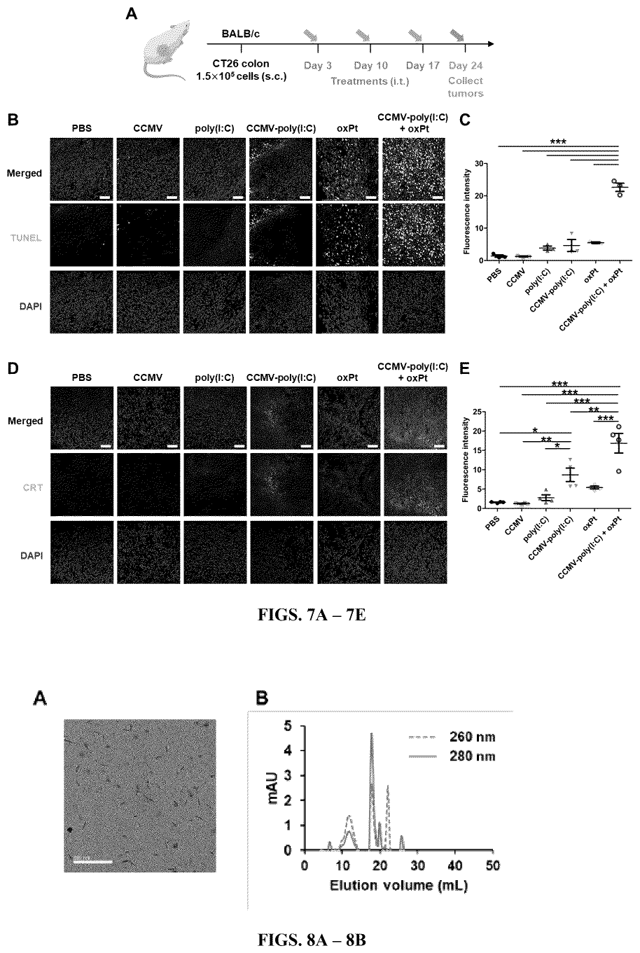

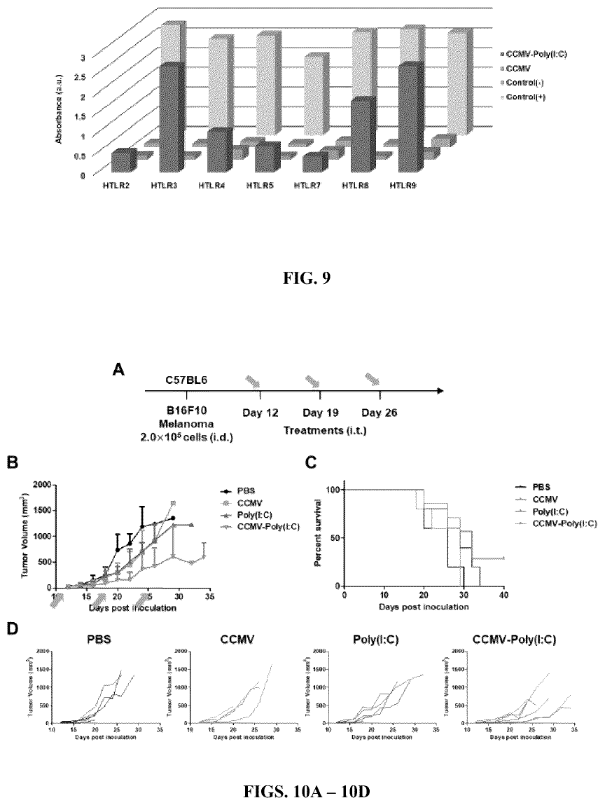

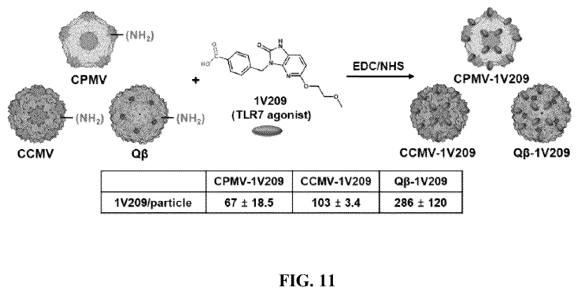

The same washes were analyzed by flow cytometry to detect activated CD4+ and CD8⁺ cells (FIG. 6). Cells were pre-gated on live, single lymphocytes and defined by CD45⁺ and CD3⁺ expression before further gating to identify CD4⁺ and CD8⁺ T cells. Although CCMV-poly(I:C) increased the infiltration of T cells compared to CCMV and poly(I:C) alone, CCMV-poly(I:C)+oxPt induced the most prolific infiltration of CD4+ and CD8+ cells (FIG. 6B). Applicant also used an ELISPOT assay to evaluate the production of IL-4 and IFN-γ from splenocytes, which play major roles in regulating immune responses (FIG. 6C). In agreement with the MSD and flow cytometry data, CCMV-poly(I:C)+oxPt enhanced the levels of IL-4 and IFN-γ more than any other treatment, confirming the synergistic immunogenic effect of the immunomodulatory VLP and oxaliplatin. It should be noted that T cell depletion studies and analysis of T cell specificity are required to further delineate the role of T cell responses and whether the treatment is T cell dependent.

Enhanced Immunogenic Cell Death Effect of CCMV-Poly(I:C) in Combination with Oxaliplatin

Applicant verified the anti-tumor mechanism of CCMV-poly(I:C)+oxPt histologically using mice bearing CT26 tumors induced by s.c. inoculation. Mice were treated i.t. three times with CCMV, poly(I:C), CCMV-poly(I:C) or oxPt. On day 24, tumor-bearing mice were euthanized, and tumors were collected and stained using the TUNEL assay to detect apoptotic cells or an anti-CRT antibody to detect ICD events (FIG. 7A). TUNEL fluorescence was not detected in tumor tissues treated with PBS or CCMV, indicating no anti-tumor activity, whereas the CCMV-poly(I:C) treatment enhanced apoptosis in the tumors compared to free poly(I:C) at the same dose (FIG. 7B, FIG. 7C). Apoptosis was enhanced further in the CCMV-poly(I:C)+oxPt combined treatment compared to the CCMV-poly(I:C) or oxPt treatments alone, with CCMV-poly(I:C)+oxPt showing 5-fold and 5.2-fold increase in apoptosis vs. CCMV-poly(I:C) or oxPt, respectively. The histology analysis thus confirms the synergistic anti-tumor effect.

Oxaliplatin causes immunogenic cell death (ICD) [24], a form of cell death that stimulates the immune system through release and presentation of danger signals or damage-associated molecular patterns (DAMPs), such as ATP, calreticulin (CRT), HMGB1, and heat-shock proteins [25]. To probe whether DAMPs are involved, Applicant imaged CRT. While CCMV-poly(I:C) slightly increased the CRT exposure compared to CCMV and poly(I:C), the combination of CCMV-poly(I:C)+oxPt significantly increased the level of CRT exposure, which is 2.7-fold and 2.5-fold increase compared to CCMV-poly(I:C) or oxPt, respectively. (FIG. 7D, FIG. 7E).

### Experimental Discussion

Applicant demonstrated that the TLR3 agonist poly(I:C) can be encapsulated within CCMV particles (CCMV-poly(I:C) VLPs) and that the resulting formulation is stable and intact in vitro and in vivo. The CCMV-poly(I:C) VLPs were immunogenic in vitro, as determined by their ability to stimulate the production of SEAP by RAW-Blue macrophages, whereas the components supplied separately had no significant effect. The therapeutic impact of CCMV-poly(I:C) alone and combined with oxaliplatin was assessed in vivo using two CT26 colon carcinoma models. CCMV alone had no effect whereas poly(I:C) and oxaliplatin were able to induce weak to moderate immune responses that slightly inhibited tumor growth and/or improved survival. The CCMV-poly(I:C) VLPs were more efficacious than either of their components, but the most potent effects in terms of both tumor inhibition and survival in all three models were achieved by the combination of CCMV-poly(I:C) VLPs and oxaliplatin. The underlying mechanism involves the infiltration and activation of CD4+ and CD8+ cells and the production of IL-4 and IFN-7 in the TME, indicating a synergistic immunogenic effect. The combined treatment also enhanced the rates of apoptosis and ICD, confirming the more potent anti-tumor response. Additional research is required to understand the synergistic mechanisms in more detail, but our results support earlier studies based on CPMV, PapMV and PVX indicating that the intratumoral injection of plant VLPs induces a localized immune response that involves the attraction of TILs and the secretion of cytokines and chemokines that convert the immunosuppressive TME into an immunostimulated phenotype. The interplay between innate and adaptive immunity results in a systemic response and the establishment of immune memory to ensure that residual cancer cells are eliminated. While the VLPs act upon the immune system, the co-administration of a separate antineoplastic drug attacks the cancer cells directly, weakening them and making them less likely to evade the immune response. The two components therefore act synergistically by acting against different targets, increasing the overall potency of the anti-tumor effect.

### Example 2: TLR Agonists Delivered by Plant Virus and Bacteriophage Nanoparticles for Cancer Immunotherapy

Toll-like receptors (TLRs) are promising targets in cancer immunotherapy due to their role in activating the immune system, therefore, various small-molecule TLR agonists have been tested in the clinic. However, clinical use of TLR agonists is hindered by their non-specific side effects and poor pharmacokinetics. To overcome these limitations, Applicant used plant viral nanoparticles (VNPs) and bacteriophage virus-like particles (VLPs) as drug delivery systems. Applicant conjugated TLR3 or TLR7 agonists to cowpea mosaic virus (CPMV) VNPs, cowpea chlorotic mottle virus (CCMV) VNPs, and bacteriophage Qβ VLPs. The conjugation of TLR7 agonist, 2-methoxyethoxy-8-oxo-9-(4-carboxybenzyl)adenine (1V209), resulted in the potent activation of immune cells and promoted the production of pro-inflammatory cytokine interleukin 6. Applicant found that 1V209 conjugated to CPMV, CCMV and Qβ reduced tumor growth in vivo and prolonged the survival of mice compared to those treated with free 1V209 or a simple admixture of 1V209 and viral particles. Nucleic acid-based TLR3 agonist, polyinosinic acid and polycytidylic acid (poly(I:C)), was also delivered by CPMV VNPs, resulting in enhanced mice survival. The data shows that coupling and co-delivery are required to enhance the anti-tumor efficacy of TLR agonists. The delivery of 1V209 or poly(I:C) conjugated to VNPs/VLPs probably enhances their efficacy due to the multivalent presentation, prolonging of tumor residence time, and targeting of innate immune cells mediated by the VNP/VLP carrier.

Toll-like receptors (TLRs) are important pattern recognition receptors (PRRs) that detect pathogen-associated molecular patterns (PAMPs) expressed on antigen-presenting cells such as dendritic cells, macrophages, neutrophils, natural killer cells, T cells and B cells¹. Following the recognition of PAMPs, TLRs signal through four cytosolic Toll-interleukin-1 receptor (TIR) domain-containing adaptor proteins (TIRAPs): myeloid differentiation primary response 88 (MyD88), MyD88 adaptor-like protein (MAL), TIR domain-containing adapter-inducing interferon-β (TRIF), and TRIF-related adaptor molecule (TRAM). This results in the activation and translocation of the nuclear factor kappa B transcription factor (NF-κB), interferon (IFN) regulatory factors and/or mitogen-activated protein kinases (MAPKs) that regulate IFN gene expression². TLR-based immunotherapy has been extensively investigated as either a single or combinational approach because it triggers a strong immune response³⁻⁵. Accordingly, TLR agonists have been evaluated as promising vaccine adjuvants for cancer treatment.

The TRL7 agonist 2-methoxyethoxy-8-oxo-9-(4-carboxybenzyl)adenine (1V209) is a synthetic molecule developed as an alternative to imiquimod, and the only TRL7 ligand currently approved by the US Food & Drug Administration (FDA)^{5,6}. Although 1V209 is well known for its anti-tumor effects and immunogenicity, clinical application is limited by its low solubility and stability, and potent cytokine production following systemic administration, resulting in toxicity and hyper-inflammation^{7,8}. The TLR3 agonist polyinosinic acid and polycytidylic acid (poly(I:C)) is a double-stranded RNA recognized by endosomal TLR3, which subsequently regulates melanoma differentiation-associated protein 5 (MDA5) and retinoic acid-inducible gene-I protein (RIG-I), ultimately triggering IFN signaling⁹. Poly(I:C) has been investigated as a potent immunostimulant or adjuvant for cancer treatment. Despite of the potential efficacy of nucleic acid derivatives, their applications are limited due to rapid degradation by nucleases and toxicity caused by the off-target toxicity¹⁰. Therefore, various drug delivery platforms have been investigated to overcome the shortcomings of free drug administration and to amplify therapeutic efficacy using a co-delivery system.

Applicant developed TLR agonist co-delivery platforms to boost systemic anti-tumor immunity using CPMV, CCMV and Qβ. The anti-tumor efficacy and immunogenicity of 1V209 conjugated to CPMV, CCMV and Qβ was tested in experimental B16F10 melanoma and CT26 colon cancer models. TLR3 agonist, poly(I:C), was also conjugated to CPMV particles to induce synergy between poly(I:C) and CPMV was also tested in the B16F10 melanoma model.

### Materials and Methods

Preparation of CPMV, CCMV, and Qβ. CPMV and CCMV were produced in black-eyed pea No. 5 plants as previously described^{24,25}. Qβ was expressed in Escherichia coli BL21 (D3) cells as previously reported²⁶. All viral particles were purified by ultracentrifugation in a sucrose gradient. The concentrations of CPMV and CCMV were determined by ultraviolet-visible light (UV-vis) absorbance spectrophotometry with coefficients of 8.1 and 5.64 mg⁻¹ mL cm⁻¹, respectively. The concentration of Qβ was verified using a Pierce BCA protein assay kit (Thermo Fisher Scientific).

Bioconjugation of CPMV, CCMV and Qβ. CPMV and Qβ were suspended in 10 mM potassium phosphate (KP) buffer (pH 7.0) whereas CCMV was suspended in 10 mM N-2-hydroxyethylpiperazine-N′-2-ethanesulfonic acid (HEPES) buffer (pH 7.4). 2400 molar excess of free 1V209 (MedChem Express) was activated with N-hydroxysuccinimide (NHS) and 1-ethyl-3-(3-dimethylaminopropyl)carbodiimide (EDC) in dimethyl sulfoxide (DMSO) for 2 h before mixing with CPMV, CCMV and Qβ suspensions and incubating for 2 h at room temperature (RT). The particles were then purified using PD MidiTrap G-25 columns (Cytiva) and 0.5-mL 100-kDa molecular weight cut off (MWCO) spin filters (EMD Millipore). The amount of 1V209 conjugated to CPMV, CCMV and Qβ was calculated by subtracting the concentration of 1V209 remaining in the supernatant from the total added to the reaction (determined by measuring the absorbance at 283 nm). For poly(I:C) conjugation, 2000 molar excess of poly(I:C) (Invivogen) was activated with EDC and 1-methylimidazole (MeIm) in nuclease-free water for 30 min at RT before mixing with CPMV and incubating for 2 h at RT. Excess reagent was removed by ultracentrifugation (52,000 g, 2 h, 4° C.) and the supernatant was collected to determine the conjugation efficiency. The concentration of poly(I:C) in the supernatant was determined by measuring the absorbance at 266 nm. using UV-vis. The amount of poly(I:C) conjugated to the particles was determined by subtracting the unconjugated fraction from the total poly(I:C). For cyanine 5 (Cy5) labeling reactions, CPMV, CCMV and Qβ were mixed with 700 molar excess of sulfo-Cy5 NHS ester (Lumiprobe) for 2 h at RT. The reaction mixtures were purified on a 30% (w/v) sucrose cushion by ultracentrifugation (52,000 g, 2 h, 4° C.) to remove excess Cy5 dye. The conjugation of Cy5 was quantified based on the dye-to-VNP/VLP ratio and the Beer-Lambert law (molecular weight of CPMV=5.6×106 g mol⁻¹, CCMV=2×10⁴ g mol⁻¹, Qβ=3.6×104 g mol⁻¹, Cy⁵=747 g mol⁻¹). The number of Cy5 dyes per particle was determined by UV-vis spectroscopy based on the specific extinction coefficients (CPMV_{ε260}=8.1 mg⁻¹ mL cm⁻¹, CCMV_{ε260}=5.87 mg⁻¹ mL cm⁻¹, Qβ_{ε260}=8.1 mg⁻¹ mL cm⁻¹, Cy5_{ε647}=271,000 mg⁻¹ mL cm⁻¹). All native and conjugated CPMV, CCMV and Qβ particles were stored at 4° C. until needed for further analysis.

Gel Electrophoresis. CPMV and Qβ particles (10 g) were analyzed by 0.8% (w/v) agarose native gel electrophoresis in Tris-acetate-EDTA (TAE) buffer for 30 min at 120 V and RT. CCMV particles were analyzed using the same method but in virus electrophoresis buffer (0.1 M sodium acetate, 1 mM EDTA, pH 5.5) for 60 min at 60 V and 4° C. as previously described¹⁸. Agarose gels were stained with GelRed nucleic acid to detect RNA and Coomassie Brilliant Blue to detect protein. The gels were imaged using an Alphalmager System under UV light, white light and MultiFluor red light for visualization of RNA, protein, and Cy5, respectively.

Transmission Electron Microscopy (TEM). CPMV, CCMV and Qβ particles were suspended at 1 mg mL⁻¹ in deionized water and deposited onto Formvar carbon film-coated copper TEM grids (Ted Pella) for 2 min, followed by washing twice with deionized water for 1 min. The TEM grids were coated with 2% (w/v) uranyl acetate for 2 min. TEM images were acquired using a JEOL JEM-1400Plus microscope.

Dynamic Light Scattering (DLS). CPMV, CCMV and Qβ particles were dispersed at a concentration of 0.1 mg mL⁻¹ in 0.1 M KP buffer for CPMV and Qβ, and 0.1 M sodium acetate buffer (pH 4.8) for CCMV. The size was measured on a Zetasizer Nano ZSP/Zen5600 (Malvern Panalytical).

Size Exclusion Cromatography (SEC). CPMV, CCMV and Qβ particles were prepared at a concentration of 3 mg mL⁻¹ and loaded onto a Superose 6 Increase column mounted on an AKTA pure chromatography system (Cytiva). The flow rate was set to 0.5 mL min⁻¹ in 0.1 M KP buffer for CPMV and Qβ or 0.1 M sodium acetate buffer for CCMV. The absorbance was recorded at 260 and 280 nm, and fluorescent particles were also measured at 647 nm to detect Cy5.

Cell Studies. B16F10 (ATCC CRL-6475, mouse skin melanoma) and CT26 (ATCC, mouse colon cancer) cells were cultured in Dulbecco's modified Eagle's medium (DMEM) and Roswell Park Memorial Institute (RPMI) 1640 medium, respectively, each supplemented with 10% (v/v) fetal bovine serum (FBS; Atlanta Biologicals) and 1% v/v) penicillin/streptomycin (P/S; Thermo Fisher Scientific). RAW-Blue cells (Invivogen) were grown in DMEM supplemented with 10% (v/v) FBS, 1% (v/v) P/S, 50 mg mL⁻¹ normocin and 100 mg mL⁻¹ zeocin, and were maintained according to supplier's introductions. RAW264.7 (ATCC) macrophages were grown in DMEM supplemented with 10% (v/v) FBS and 1% (w/v) P/S. All cells were maintained at 37° C. in a humidified incubator with a 5% CO2 atmosphere.

RAW-Blue Assay. RAW-Blue cells were seeded in 96-well plates (1×10⁵ cells/well) and treated with 1 g of native or 1V209-conjugated CPMV, CCMV and Qβ (as well as controls). After 24 h, 20 μL of the supernatant from each well was mixed with 180 μL Quanti-Blue solution (Invivogen) and incubated for 6 h at RT before recording the absorbance at 630 nm on a Tecan plate reader.

Enzyme-Linked Immunosorbent Assay (ELISA). RAW264.7 cells were seeded into 24-well plates (5×10⁵ cells/well) and treated with 5 g of native or 1V209-conjugated CPMV, CCMV and Qβ. After 24 h, the supernatants were collected from each well and the quantities of interleukin (IL)-6, IL-12 and IFN-g were determined using the appropriate ELISA kits (Thermo Fisher Scientific)

Animal Studies. C57BL/6 and BALB/c mice (The Jackson Laboratory) were used for animal experiments carried out in accordance with the guidelines set out by the Institutional Animal Care and Use Committee of the University of California, San Diego. For the melanoma model, C57BL/6 mice were inoculated intradermally (i.d.) with B16F10 cells (2×10⁵ cells in 20 μL of PBS) in the left flank. The mice then received three treatments with native or conjugated CPMV, CCMV and Qβ particles starting 9 days post-inoculation with 1 week intervals between injections. Animal survival and tumor volume were recorded every 2 days, and the tumor volume was calculated using the following equation: tumor volume=(tumor width²×tumor length)/2. For the colon cancer model, BALB/c mice were injected intraperitoneally (i.p.) with CT26 cells (5×10⁵ cells in 100 μL PBS). The mice then received three treatments with native or conjugated CPMV, CCMV and Qβ particles starting on day 3 with 1 week intervals between injections. Applicant measured the circumference and body weight of the mice every 2 days.

Fluorescence Imaging. Mice were inoculated i.d. with B16F10 cells (2×10⁵ cells in 20 μL of PBS). When the tumors had grown to −100 mm³, Cy5-conjugated CPMV, CCMV and Qβ particles (100 g in 20 μL of PBS) were injected into the tumors. Fluorescent images were acquired using an in vivo imaging system (IVIS, Xenogen) and longitudinal imaging was carried out for 7 days.

Statistical Analysis. Data from in vitro and in vivo studies were analyzed in GraphPad Prism v8 and are presented as mean±standard deviations. For multiple comparisons, statistical significance was determined using one-way ANOVA with a post hoc Tukey's HSD test. For survival data, curves were generated according to the Mantel-Cox test and were compared statistically using the log-rank test. Significance was assigned at P<0.05.

### Results and Discussion

### Characterization of 1V209-Conjugated Viral Particles

To address the non-specific side effects and poor pharmacokinetics of 1V209 administered as a free drug^{5,8}, Applicant conjugated the TRL7 agonist to plant VNPs and bacteriophage VLPs as drug delivery systems (FIG. 11). Applicant also conjugated the virus particles to poly(I:C) and Cy5 for the evaluation of combination therapy and particle stability. CPMV, CCMV and Qβ were prepared and purified as previously described^{25,26}, and exposed lysine (Lys) residues were reacted with 1V209 using NHS/EDC chemistry. EDC activates the carboxylic acid group of 1V209, and NHS then forms a stable amine-reactive sulfo-NHS ester that binds the exposed Lys on the viral surfaces. The conjugated particles are hereafter described as CPMV−1V209, CCMV−1V209 and Qβ−1V209.

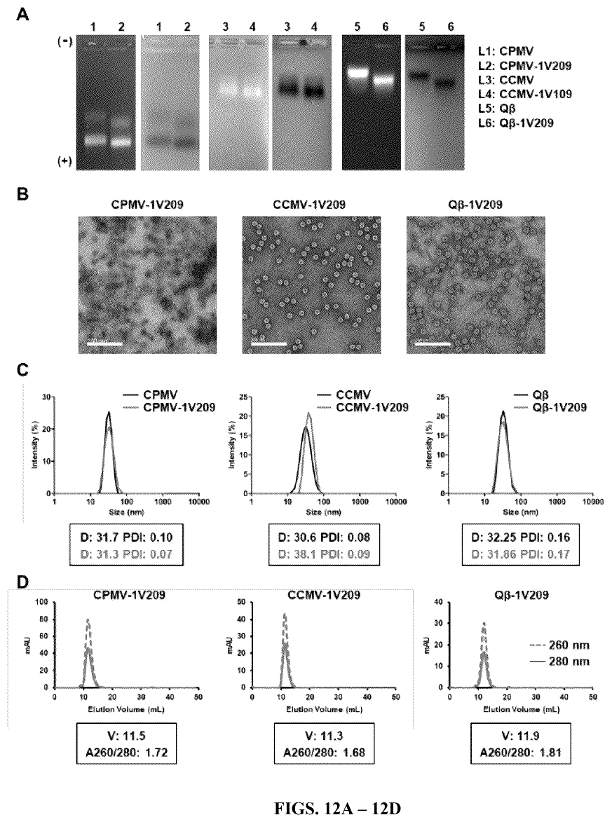

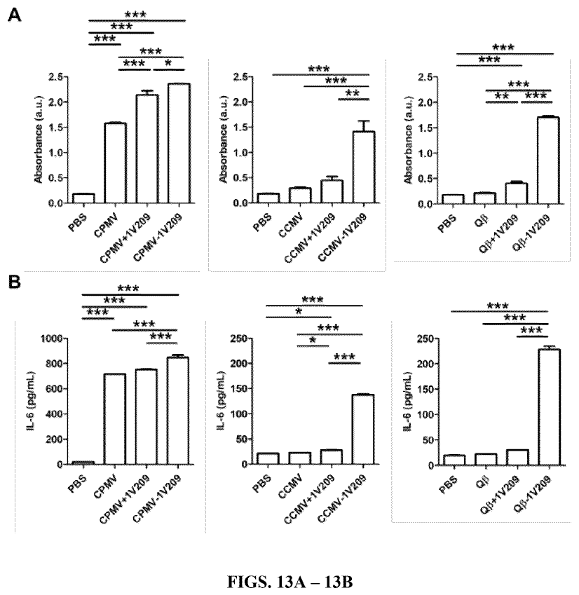

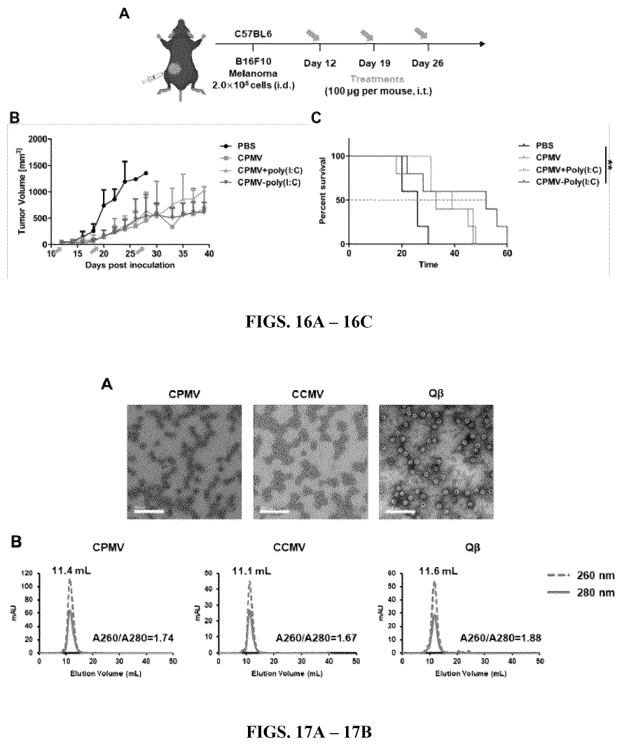

The conjugation efficiency was determined by subtracting the concentration of free 1V209 remaining after the reaction from the starting 1V209 concentration, revealing that CPMV, CCMV and Qβ carried 67, 103 and 286 1V209 molecules, respectively. Conjugation was confirmed by agarose gel electrophoresis (FIG. 12A). The electrophoretic mobility of CPMV 1V209, CCMV−1V209 and Qβ−1V209 towards the anode increased with the number of conjugated 1V209 molecules (1V209 conjugates to Lys side chains and each conjugation therefore replaces a positive charge). The conjugated particles remained intact, and their morphology was similar to the corresponding native particles, as confirmed by TEM (FIG. 12B and FIG. 17A). Likewise, DLS experiments showed that the native and conjugated particles had similar average diameters of ˜30 nm with no evidence of aggregation (FIG. 12C). The SEC elution profiles including absorbance ratios of CPMV−1V209, CCMV−1V209 and Qβ−1V209 at 260/280 nm as well as the volume of elution were also similar to the native particles, further confirming their integrity and purity (FIG. 12D and FIG. 17B).

### Immunogenicity of 1V209-Conjugated Viral Particles

Applicant investigated the immunogenicity of the 1V209-conjugated VNPs/VLPs using RAW-Blue macrophages, which express various PRRs and a secreted embryonic alkaline phosphatase (SEAP) reporter allowing us to monitor NF-κB activation using a Quanti-Blue assay (FIG. 13A). The SEAP level increased sharply in response to CPMV, which was ˜5.5-fold higher than CCMV and ˜7.4-fold higher than Qβ. This is consistent with prior work that indicates that CPMV is more immunogenic than the other particles due to its multi-pronged ability to trigger TLR2, TLR4 and TLR7 signaling¹². The admixture of CPMV+1V209 produced ˜1.3-fold more SEAP than CPMV alone, but admixtures of the other particles with 1V209 did not significantly increase the SEAP activity compared to the native particles. In contrast, CPMV−1V209, CCMV−1V209 and Qβ−1V209 increased SEAP activity by ˜1.1-fold, ˜3.2-fold and ˜4.2-fold, respectively, compared to the admixtures of each native particle+1V209.

Next, Applicant compared the effect of 1V209-conjugated particles on TLR7-mediated IL-6 secretion (FIG. 13B). IL-6 levels induced by native CPMV were ˜21-fold higher compared to native CCMV and ˜32-fold higher compared to native Qβ, which was consistent with the RAW-Blue assay. Admixtures of CPMV, CCMV and Qβ with 1V209 activated RAW 264.7 cells at a similar level to each native particle, indicating no synergistic effects with 1V209. In contrast, CPMV−1V209, CCMV−1V209 and Qβ−1V209 significantly increased the IL-6 levels in RAW264.7 cells by ˜1.2-fold, ˜1.7-fold and ˜7.4-fold, respectively, compared to the corresponding admixture groups (FIG. 13B). Together, these data indicate that 1V209 must be conjugated to and not co-delivered with the viral particles in order to achieve enhanced immunogenicity.

### Therapeutic Efficacy of 1V209-Conjugated Virus Particles In Vivo

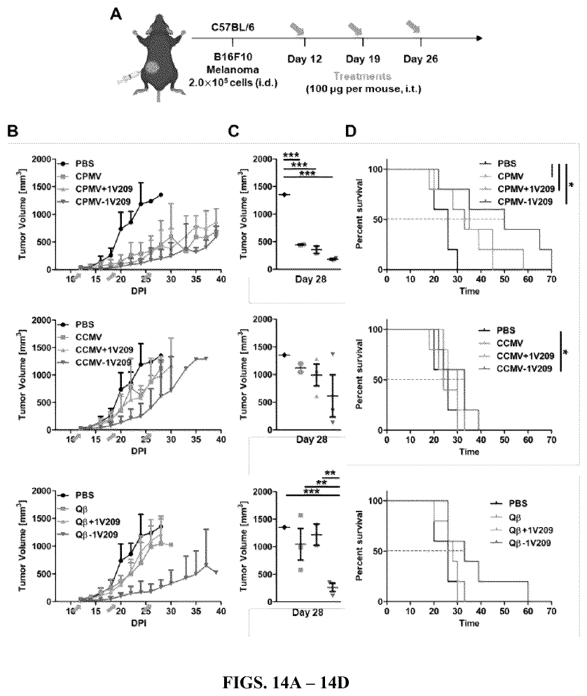

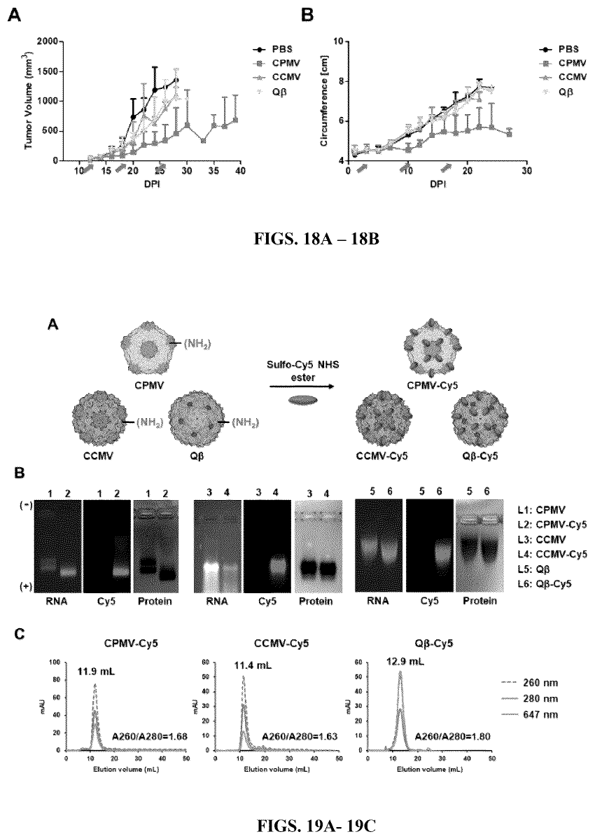

The therapeutic efficacy of the 1V209-conjugated particles was evaluated in dermal melanoma and colon cancer models. For the dermal melanoma model, female C57BL/6 mice were inoculated with a B16F10 cells (2×10⁵ cells, i.d). Particles were injected when the tumor reached a volume of ˜30 mm³ (˜day 12) for a total of 3 weekly injections (FIG. 14A). In mice injected with native CPMV, tumor growth was reduced by 66.5% on day 28 compared to the PBS control group (FIGS. 14B, 14C). CPMV performed better than CCMV (17.3% growth reduction) and Qβ (22.75% growth reduction) (FIG. 18A), which is consistent with the unique anti-cancer immunity induced by CPMV as previously reported¹². The admixture of CPMV+1V209 inhibited tumor growth by 68%, which was similar to CPMV alone. However, CPMV−1V209 inhibited tumor growth by 85% (FIG. 14B, FIG. 14C) and increased the median survival rate to 50 days (FIG. 14D), which was an improvement compared to other groups (PBS control 26 days, CPMV 33 days and CPMV+1V209 35 days). CCMV and the admixture of CCMV+1V209 inhibited tumor growth on day 28 by 17.2% and 26.8%, respectively (FIG. 14B, FIG. 14C), and resulted in median survival rates of 24 and 30 days, respectively, indicating no significant anti-tumor efficacy. However, CCMV−1V209 inhibited tumor growth by 54.6% on day 28 and prolonged the median survival period to 33 days (FIG. 14D). Similarly, Qβ and the admixture of Qβ+1V209 inhibited tumor growth on day 28 by 22.7% and 10%, respectively (FIG. 14B, FIG. 14C), and the median survival periods were 27 and 28 days, respectively (FIG. 14D). However, Qβ−1V209 showed a potent anti-cancer effect, inhibiting tumor growth by 80% and prolonging the median survival period to 33 days. When comparing CCMV and Qβ, neither particle was efficacious as a solo therapy, but Qβ−1V209 performed better than CCMV−1V209. Without wishing to be bound by any particular theory, Applicant presumes this was because more 1V209 molecules were conjugated to each Qβ particle (FIG. 11).

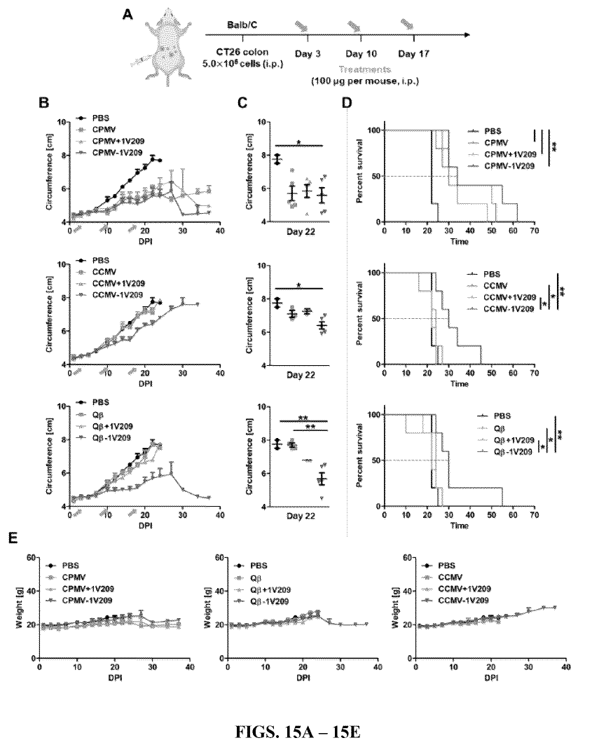

In the CT26 colon cancer model, BALB/c mice were challenged i.p. with CT26 cells and treated with particles 3 days post-inoculation (FIG. 15A). CPMV inhibited tumor growth by 26.5% compared to the PBS-treated controls on day 22 and was significantly more effective than CCMV and Qβ (FIG. 18B). The admixture of CPMV+1V209 and the CPMV−1V209 particles did not show any synergistic inhibition of tumor growth compared to native CPMV, with 24.6% and 28.5% inhibition on day 22, respectively (FIG. 15B, FIG. 15C). The median survival periods for the native CPMV, admixture and conjugated particles were 30, 30 and 34 days, respectively (FIG. 15D). The similar performance of the three CPMV-based treatments probably reflects the already potent anti-tumor effect of native CPMV in the CT26 colon cancer model¹². CCMV and the admixture of CCMV+1V209 inhibited tumor growth by 8.4% and 6.5% on day 22, respectively, which did not differ significantly from the PBS control (FIG. 15B, FIG. 15C). Similarly, the median survival periods of the PBS control, CCMV and CCMV+1V209 admixture were 22, 20 and 22 days, respectively (FIG. 15D), confirming that these formulations showed no anti-tumor efficacy. However, CCMV−1V209 reduced tumor growth by 17.5% compared to the PBS control on day 22 and prolonged the survival to 30 days. Finally, Qβ−1V209 reduced tumor growth by 21% compared to the PBS control, whereas the native Qβ particles (0.65% reduction) and admixture with 1V209 (12.2% reduction) did not show a significant effect. CCMV−1V209 was less effective than Qβ−1V209, again presumably due to the number of conjugated drug molecules. The accumulation of blood and other body fluids in the peritoneal space increased the circumference and body weight of the treated mice (FIG. 15B, FIG. 15E).

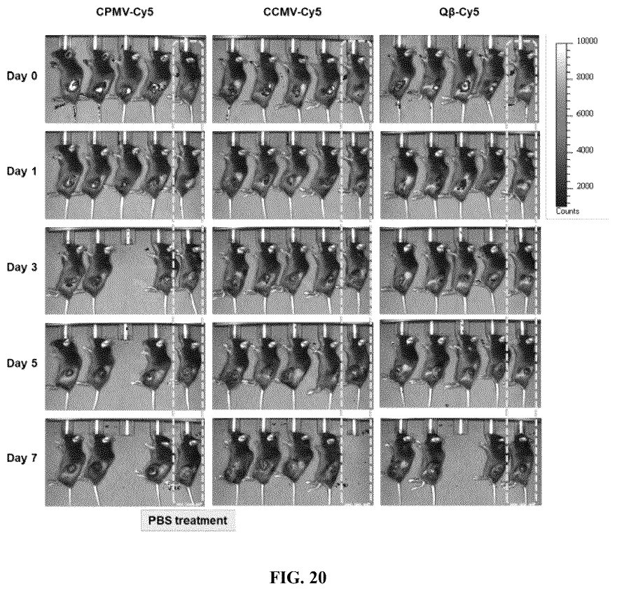

Applicant also assessed the retention of CPMV, CCMV and Qβ in tumors after i.t. administration to understand the relationship between particle stability and therapeutic efficacy. Long-term tumor retention and multivalent/polyvalent binding of TLRs are important factors for in situ vaccination, which determine therapeutic efficacy²⁷. For live animal IVIS imaging, CPMV, CCMV and Qβ were labeled with Cy5, resulting in CPMV-Cy5, CCMV-Cy5 and Qβ−Cy5 particles. These were characterized by agarose gel electrophoresis and SEC to confirm their integrity (FIG. 19B) before injection into mice bearing B16F10 tumors with a volume of at least ˜100 mm³. The i.t. injection of CPMV-Cy5, CCMV-Cy5 and Qβ−Cy5 resulted in similar retention times exceeding 7 days in all tumors, suggesting that the observed differences in therapeutic efficacy−reflect the conjugation efficiency rather than the stability of each type of particle (FIG. 20).

### Characterization, Immunogenicity and Efficacy of Poly(I:C)-Conjugated Viral Particles

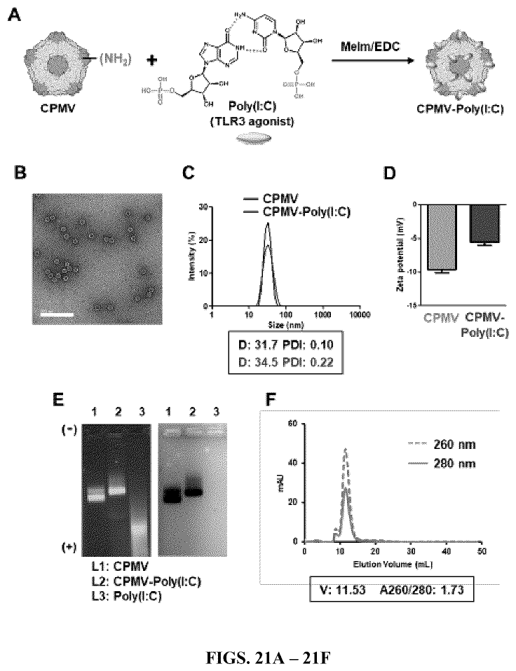

Applicant previously demonstrated that CPMV exerts its potent immunogenicity by activating TLR2, TLR4 and TLR7¹¹. The combination of CPMV and 1V209 showed moderate improvement to native CPMV, which may be explained by the fact that CPMV already signals through TLR7. While conjugation of 1V209 to CPMV did not increase efficacy, therapeutic effect was more potent for CCMV−1V209 and Qβ−1V209 vs. their non-conjugated versions when tested in the B16F10 and CT26 models (FIG. 14 and FIG. 15). CCMV and Qβ do not signal through TLR7. Therefore, to determine whether synergy could be achieved by combining CPMV with TLRs other than TLR2, TLR4 and TLR7, Applicant combined CPMV with the TLR3 agonist poly(I:C). The amine residues in CPMV were conjugated to poly(I:C) using EDC and MeIm chemistry (FIG. 21A). The 5′-end phosphate group of poly(I:C) was functionalized by EDC and MeIm to form a phosphoramidate in the presence of the primary amine group of CPMV Lys residues. The conjugation efficiency was measured by subtracting the free poly(I:C) remaining after conjugation from the total initial amount, revealing the presence of ˜25 poly(I:C) strands per particle. CPMV and the conjugated derivative CPMV-poly(I:C) were shown to be monodisperse, icosahedral particles by TEM, and DLS indicated particle diameters of ˜31.7 nm (PDI=0.1) and ˜34.5 nm (PDI=0.22), respectively (FIG. 21B, FIG. 21C). The surface charge of CPMV was recorded as ˜13.5 mV, which increased to −5.6 mV following conjugation (FIG. 21D). Accordingly, the poly(I:C)-conjugated CPMV were less mobile in agarose gels than the native CPMV particles (FIG. 21E). The underlying factor that contributes to the lower negative zeta potential is counterintuitive because poly(I:C) carries negative charge, nevertheless zeta potential and native gels show consistent results. SEC confirmed the structural integrity of CPMV and CPMV-poly(I:C), with the same elution volume of ˜11 mL and an absorbance ratio at 260/280 nm of ˜1.7, indicating intact capsid proteins and RNAs (FIG. 21F).

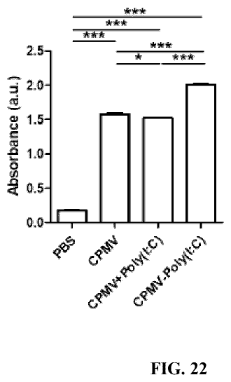

Applicant next investigated the immunogenicity of CPMV-poly(I:C) using RAW-Blue cells (FIG. 22). Native CPMV and the CPMV+poly(I:C) admixture activated RAW-Blue cells to a similar extent, but the CPMV-Poly(I:C) particles increased the amount of SEAP by ˜1.3-fold compared to CPMV. To investigate the potential synergistic effect of CPMV and poly(I:C) in vivo, B16F10 cells were injected i.d. into the flank of C57BL/6 mice. When the tumor reached a volume of ˜30 mm³ (˜day 12), Applicant administrated native, admixed or conjugated particles (FIG. 16A). CPMV-poly(I:C) did not inhibit tumor growth to a significantly greater extent than the native particles (FIG. 16B) but prolonged the median survival period to 52 days, compared to 33 days for the native particles and 39 days for the CPMV+poly(I:C) admixture (FIG. 16C). This data may indicate that the addition of further TLR agonists to CPMV does not confer enhanced anti-tumor potency potentially due to the similar signaling pathways between TLRs. Adding TLR3 does not do much because the maximum signaling through that pathways has already been reached. In other work, Applicant will focus on other combinations. For example, Applicant has previously demonstrated synergy with NK agonists²⁸, checkpoint inhibitors²⁹, radiation therapy³⁰, cryoablation³¹, and chemotherapy³².

### Experimental Conclusions

TLR agonists are powerful adjuvants for cancer immunotherapy. The potential of the plant viruses CPMV and CCMV as well as bacteriophage Qβ as drug delivery platforms for TLR agonists was demonstrated, which have a short biological half-life and low therapeutic efficacy when administered without a carrier. The conjugated TLR7 agonist (CPMV−1V209, CCMV-1V209 and Qβ−1V209) activated immune cells, induced the production of pro-inflammatory cytokines, and inhibited tumor growth in the B16F10 melanoma and CT26 colon cancer models. The CCMV−1V209 and Qβ-1V209 conjugates showed greater therapeutic efficacy than the admixtures of each native particle and 1V209, confirming that drug carriers are necessary to improve drug efficacy. CPMV did not show a significant synergistic effect when combined with TLR3 and TLR7 agonists. This also demonstrates that CPMV itself is a potent immunoadjuvant that may not benefit from additional TLR activation.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All references are herein incorporated in their entirety for any and all purposes.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. Throughout and without this disclosure, technical publications may be referenced by an Arabic number, the complete citations for such can be found immediately preceding the claims.

### Clauses and Embodiments

Clause 1. A method for inhibiting the growth of a mammalian cancer cell growth or stimulating the immune response of a mammalian cell, comprising contacting the cell with an effective amount of a viral nanoparticle comprising at least one TLR agonist selected from a double-stranded RNA (dsRNA) or a synthetic dsRNA that activates a TLR and a chemotherapeutic agent, thereby inhibiting the growth of the mammalian cancer cell or stimulate the immune response of the mammalian cell.

Clause 2. The method of clause 1, wherein the contacting is in vitro or in vivo.

Clause 3. A method for one or more of: (a) treating cancer in a subject in need thereof; (b) inhibiting, delaying, slowing down, or preventing relapse of cancer in a subject in need thereof; or (c) triggering or enhancing one or more of the following in a subject in need thereof; an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells, comprising administering to the subject administering an effective amount of a viral nanoparticle, wherein the nanoparticle comprises at least one TLR agonist selected from a double-stranded RNA (dsRNA) or a synthetic dsRNA capable of activating a TLR; and a chemotherapeutic agent, and thereby treating the cancer, delaying, slowing down, or preventing the relapse of cancer, or triggering or enhancing one or more of the following in a subject in need thereof: an immune response, immunostimulatory cytokines, an increase in the ratio of M1 (antitumor) to M2 (protumor) macrophages, an increase in natural killer cells (NK cells), an increase in CD4+ cells, or an increase in CD8+ cells in the subject in the subject.

Clause 4. The method of clause 3, wherein the immunostimulatory cytokine or chemokine is selected from IL-4, IL-5, IL-13, IL-15, IFN-SYMBOL, IP-10, MCP-1, MCP-1α, MCP-1β, Eotoxin, or TNF-α.

Clause 5. The method of any of clauses 1-4, wherein the viral nanoparticle is derived from a Cowpea chlorotic mottle virus (CCMV), a Cowpea mosaic virus (CPMV), a Physalis mottle virus (PhMV), a Papaya mosaic virus (PapMV), a Potato virus X (PVX), or a bacteriophage Qbeta (Qβ).

Clause 6. The method of any of clauses 1-5, wherein the nanoparticle comprises a capsid protein, further optionally from CCMV, CPMV, or a combination thereof.

Clause 7. The method of any of claims clause 1-6, wherein the TLR is TLR3, optionally wherein the TLR3 agonist is poly(I:C).

Clause 8. The method of clause 7, wherein the chemotherapeutic agent is selected from 5-fluorouracil, methyl-CCNU, oxaliplatin, irinotecan, mitomycin, cytarabine, doxorubicin, or cyclophosphamide.

Clause 9. The method of clause 8, wherein the chemotherapeutic agent comprises or consists essentially of oxaliplatin.

Clause 10. The method of any of clauses 3-9, wherein the cancer is selected from a sarcoma, a carcinoma or a blood cancer, optionally a colon cancer, a rectal cancer, or a melanoma, and further optionally a Stage I, Stage II, a Stage III or a Stage IV cancer.

Clause 11. The method of any of clauses 3-10, wherein the subject in need is a mammal, optionally wherein the mammal is selected from a human, an ape, a gibbon, a chimpanzee, an orangutan, a monkey, a macaques, a dog, a cat, a horse, a cow, a goat, a sheep, a pig, a mouse, a rabbit, or a guinea pig.

Clause 12. The method of clause 11, wherein the subject in need is a cancer patient.

Clause 13. The method of any of clauses 3-12, wherein the nanoparticle is administered intraperitoneally or subcutaneously.

Clause 14. The method of clause 13, wherein the nanoparticle and the chemotherapeutic agent are administrated in one dose or in at least two separate doses.

Clause 15. The method of clause 14, wherein the nanoparticle and the chemotherapeutic agent are administered concurrently or sequentially.

Clause 16. A composition comprising (a) a viral nanoparticle comprising at least one toll-like receptor (TLR) agonist selected from a double-stranded RNA (dsRNA) or a synthetic dsRNA capable of activating a TLR; and (b) at least one chemotherapeutic agent, and optionally a carrier, and further optionally a detectable label, wherein the TLR is selected from TLR1, TLR2, TLR3, TLR4, TLR5, TLR6, TLR7, TLR8, or TLR9, optionally wherein the virus is CPMV, CMMV, or Qβ.

Clause 17. The composition of clause 16, wherein the chemotherapeutic agent is selected from 5-fluorouracil, methyl-CCNU, oxaliplatin, irinotecan, mitomycin, cytarabine, doxorubicin, or cyclophosphamide.

Clause 18. The composition of clause 17, wherein the chemotherapeutic agent comprises or consists essentially of oxaliplatin.

Clause 19. The composition of any of clauses 16-18, wherein the TLR is TLR3.

Clause 20. The composition of clause 19, wherein the TLR is TLR3, the TLR agonist is polyinosinic:polycytidylic acid (poly(I:C)), optionally wherein the chemotherapeutic agent comprises or consists essentially of oxaliplatin.

Clause 21. A method to inhibit the growth of a mammalian cancer cell or stimulate the immune response of a mammalian cell, comprising contacting the cell the composition of any of clauses 16-20, thereby inhibiting the growth of the cancer cell or stimulating the immune response of a mammalian cell.

Clause 22. The method of clause 21, wherein the contacting is in vitro or in vivo.

Clause 23. A method for: (a) treating cancer in a subject in need thereof; (b) inhibiting, delaying, slowing down, or preventing relapse of cancer in a subject in need thereof; or (c) triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells, comprising administering to the subject an effective amount of the composition of any of clauses 16-20, thereby treating the cancer, delaying, slowing down, or preventing the relapse of cancer, or triggering or enhancing one or more of the following in a subject in need thereof: an immune response, immunostimulatory cytokines, an increase in the ratio of M1 (antitumor) to M2 (protumor) macrophages, an increase in natural killer cells (NK cells), an increase in CD4+ cells, or an increase in CD8+ cells in the subject in the subject.

Clause 24. The method of clause 23, wherein the immunostimulatory cytokine or chemokine is selected from IL-4, IL-5, IL-13, IL-15, IFN-SYMBOL, IP-10, MCP-1, MCP-1α, MCP-1β, Eotoxin, or TNF-α.

Clause 25. The method of clauses 23-24, wherein the cancer is selected from a sarcoma, a carcinoma or a blood cancer, optionally a colon cancer, a rectal cancer, or a melanoma, and further optionally a Stage I, Stage II, a Stage III or a Stage IV cancer.

Clause 26. The method of any clauses 23-25, wherein the subject in need is a mammal, optionally wherein the mammal is selected from a human, an ape, a gibbon, a chimpanzee, an orangutan, a monkey, a macaque, a dog, a cat, a horse, a cow, a goat, a sheep, a pig, a mouse, a rabbit, or a guinea pig.

Clause 27. The method of clause 26, wherein the subject in need is a cancer patient.

Clause 28. The method of any of clauses 21-27, wherein the composition is administered intraperitoneally, subcutaneously, or intravenously.

Clause 29. A kit comprising the composition of any of clauses 16-20 and an optional instruction for use.

Clause 30. A composition comprising (a) a viral nanoparticle comprising a toll-like receptor (TLR) agonist capable of activating a TLR; and (b) a chemotherapeutic agent, and optionally a carrier, and further optionally a detectable label, wherein the TLR is selected from TLR1, TLR2, TLR3, TLR4, TLR5, TLR6, TLR7, TLR8, or TLR9, optionally wherein the virus is CPMV, CMMV, or Qβ.

Clause 31. The composition of clause 30, wherein the chemotherapeutic agent is selected from 5-fluorouracil, methyl-CCNU, oxaliplatin, irinotecan, mitomycin, cytarabine, doxorubicin, or cyclophosphamide.

Clause 32. The method of clause 30, wherein the TLR is TLR7, wherein the TLR agonist is 2-methoxyethoxy-8-oxo-9-(4-carboxybenzyl)adenine (1V209), optionally wherein the chemotherapeutic agent comprises or consists essentially of oxaliplatin.

Clause 33. The composition of clause 30, wherein the TLR is TLR3, the TLR agonist is polyinosinic:polycytidylic acid (poly(J:C)), optionally wherein the chemotherapeutic agent comprises or consists essentially of oxaliplatin.

Clause 34. A method to prevent mammalian cell growth or stimulating the immune response of a mammalian cell comprising contacting the cell the composition of any of clauses 30-33, thereby preventing the mammalian cell growth or stimulating the immune response of the mammalian cell.

Clause 35. The method of clause 34, wherein the contacting is in vitro or in vivo.

Clause 36. A method for: (a) treating cancer in a subject in need thereof; (b) inhibiting, delaying, slowing down, or preventing relapse of cancer in a subject in need thereof; or (c) triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells, comprising administering to the subject an effective amount of the composition of any of clauses 30-33, thereby treating the cancer, delaying, slowing down, or preventing the relapse of cancer, or triggering or enhancing one or more of the following in a subject in need thereof: an immune response, immunostimulatory cytokines, an increase in the ratio of M1 (antitumor) to M2 (protumor) macrophages, an increase in natural killer cells (NK cells), an increase in CD4+ cells, or an increase in CD8+ cells in the subject in the subject.

Clause 37. The method of clause 36, wherein the immunostimulatory cytokine or chemokine is selected IL-4, IL-5, IL-13, IL-15, IFN-SYMBOL, IP-10, MCP-1, MCP-1α, MCP-13, Eotoxin, or TNF-α.

Clause 38. The method of clauses 36-37, wherein the cancer is selected from a sarcoma, a carcinoma or a blood cancer, optionally a colon cancer, a rectal cancer, or a melanoma, and further optionally a Stage I, Stage II, a Stage III or a Stage IV cancer.

Clause 39. The method of any of clauses 36-38, wherein the subject in need is a mammal, optionally wherein the mammal is selected from a human, an ape, a gibbon, a chimpanzee, an orangutan, a monkey, a macaque, a dog, a cat, a horse, a cow, a goat, a sheep, a pig, a mouse, a rabbit, or a guinea pig.

Clause 40. The method of clause 39, wherein the subject in need is a cancer patient.

Clause 41. The method of any of clauses 36-40, wherein the composition is administered intraperitoneally, subcutaneously, or intravenously.

Clause 42. A kit comprising the composition of any of clauses 30-33 and an optional instruction for use.

Clause 43. A composition comprising (a) a viral nanoparticle comprising at least two toll-like receptor (TLR) agonists capable of activating a TLR; and (b) at least one chemotherapeutic agent, and optionally a carrier, and further optionally a detectable label, wherein the TLR is selected from TLR1, TLR2, TLR3, TLR4, TLR5, TLR6, TLR7, TLR8, or TLR9, optionally wherein the plant virus is CPMV, CMMV, or Qβ.

Clause 44. The composition of clause 43, wherein the at least two TLR agonists activate the same TLR or different TLRs.

Clause 45. The composition of clauses 43-44, wherein the at least two TLR agonists are 1V209 and poly(I:C).

Clause 46. The composition of clause 45, wherein the chemotherapeutic agent is selected from 5-fluorouracil, methyl-CCNU, oxaliplatin, irinotecan, mitomycin, cytarabine, doxorubicin, cyclophosphamide.

Clause 47. A method to prevent mammalian cell growth or stimulating the immune response of a mammalian cell comprising contacting the cell the composition of any of clauses 43-46, thereby preventing the mammalian cell growth or stimulating the immune response of the mammalian cell.

Clause 48. The method of clause 47, wherein the contacting is in vitro or in vivo.

Clause 49. A method for: (a) treating cancer in a subject in need thereof; (b) inhibiting, delaying, slowing down, or preventing relapse of cancer in a subject in need thereof; or (c) triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells, comprising administering to the subject an effective amount of the composition of any of clauses 43-46, and thereby treating the cancer, delaying, slowing down, or preventing the relapse of cancer, or triggering or enhancing one or more of the following in a subject in need thereof: an immune response, immunostimulatory cytokines, an increase in the ratio of M1 (antitumor) to M2 (protumor) macrophages, an increase in natural killer cells (NK cells), an increase in CD4+ cells, or an increase in CD8+ cells in the subject in the subject.

Clause 50. The method of clause 49, wherein the immunostimulatory cytokine or chemokine is selected IL-4, IL-5, IL-13, IL-15, IFN-SYMBOL, IP-10, MCP-1, MCP-1α, MCP-13, Eotoxin, or TNF-α.

Clause 51. The method of clauses 49-50, wherein the cancer is selected from a sarcoma, a carcinoma or a blood cancer, optionally a colon cancer, a rectal cancer, or a melanoma, and further optionally a Stage I, Stage II, a Stage III or a Stage IV cancer.

Clause 52. The method of any of clauses 49-51, wherein the subject in need is a mammal, optionally wherein the mammal is selected from a human, an ape, a gibbon, a chimpanzee, an orangutan, a monkey, a macaque, a dog, a cat, a horse, a cow, a goat, a sheep, a pig, a mouse, a rabbit, or a guinea pig.

Clause 53. The method of clause 52, wherein the subject in need is a cancer patient.

Clause 54. The method of any of clauses 49-53, wherein the composition is administered intraperitoneally, subcutaneously, or intravenously.

Clause 55. A kit comprising the composition of any of clauses 43-46 and an optional instruction for use.

### REFERENCES EXAMPLE 1 (AND REFERENCES WITH [BRACKETS])

[1] D. S. Chen, I. Mellman, Oncology meets immunology: the cancer-immunity cycle, Immunity 39(1) (2013) 1-10.
[2] C. J. Melief, O. J. Finn, Cancer immunology, Curr Opin Immunol 23(2) (2011) 234-6.
[3] M. A. Postow, Managing immune checkpoint-blocking antibody side effects, Am Soc Clin Oncol Educ Book (2015) 76-83.
[4] L. Hammerich, A. Binder, J. D. Brody, In situ vaccination: Cancer immunotherapy both personalized and off-the-shelf, Mol Oncol 9(10) (2015) 1966-81.
[5] K. E. Allison, B. L. Coomber, B. W. Bridle, Metabolic reprogramming in the tumour microenvironment: a hallmark shared by cancer cells and T lymphocytes, Immunology 152(2) (2017) 175-184.
[6] P. A. Beavis, C. Y. Slaney, M. H. Kershaw, D. Gyorki, P. J. Neeson, P. K. Darcy, Reprogramming the tumor microenvironment to enhance adoptive cellular therapy, Semin Immunol 28(1) (2016) 64-72.
[7] K. Li, S. Qu, X. Chen, Q. Wu, M. Shi, Promising Targets for Cancer Immunotherapy: TLRs, RLRs, and STING-Mediated Innate Immune Pathways, Int J Mol Sci 18(2) (2017).
[8] A. H. Rook, J. M. Gelfand, M. Wysocka, A. B. Troxel, B. Benoit, C. Surber, R. Elenitsas, M. A. Buchanan, D. S. Leahy, R. Watanabe, I. R. Kirsch, E. J. Kim, R. A. Clark, Topical resiquimod can induce disease regression and enhance T-cell effector functions in cutaneous T-cell lymphoma, Blood 126(12) (2015) 1452-61.
[9] D. B. Johnson, I. Puzanov, M. C. Kelley, Talimogene laherparepvec (T-VEC) for the treatment of advanced melanoma, Immunotherapy 7(6) (2015) 611-9.
[10] C. Pasare, R. Medzhitov, Toll-like receptors: linking innate and adaptive immunity, Adv Exp Med Biol 560 (2005) 11-8.
[11] M. E. Lebel, K. Chartrand, E. Tarrab, P. Savard, D. Leclerc, A. Lamarre, Potentiating Cancer Immunotherapy Using Papaya Mosaic Virus-Derived Nanoparticles, Nano Lett 16(3) (2016) 1826-32.
[12] P. H. Lizotte, A. M. Wen, M. R. Sheen, J. Fields, P. Rojanasopondist, N. F. Steinmetz, S. Fiering, In situ vaccination with cowpea mosaic virus nanoparticles suppresses metastatic cancer, Nat Nanotechnol 11(3) (2016) 295-303.
[13] K. L. Lee, A. A. Murray, D. H. T. Le, M. R. Sheen, S. Shukla, U. Commandeur, S. Fiering, N. F. Steinmetz, Combination of Plant Virus Nanoparticle-Based in Situ Vaccination with Chemotherapy Potentiates Antitumor Response, Nano Lett 17(7) (2017) 4019-4028.
[14] M. E. Lebel, J. F. Daudelin, K. Chartrand, E. Tarrab, U. Kalinke, P. Savard, N. Labrecque, D. Leclerc, A. Lamarre, Nanoparticle adjuvant sensing by TLR7 enhances CD8+ T cell-mediated protection from Listeria monocytogenes infection, J Immunol 192(3) (2014) 1071-8.
[15] C. Mao, V. Beiss, J. Fields, N. F. Steinmetz, S. Fiering, Cowpea mosaic virus stimulates antitumor immunity through recognition by multiple MYD88-dependent toll-like receptors, Biomaterials 275 (2021) 120914.
[16] C. Mao, V. Beiss, G. W. Ho, J. Fields, N. F. Steinmetz, S. Fiering, In situ vaccination with cowpea mosaic virus elicits systemic antitumor immunity and potentiates immune checkpoint blockade, J Immunother Cancer 10(12) (2022).
[17] H. Cai, S. Shukla, N. F. Steinmetz, The Antitumor Efficacy of CpG Oligonucleotides is Improved by Encapsulation in Plant Virus-Like Particles, Advanced Functional Materials (2020) https://doi.org/10.1002/adfm.201908743]
[18] J. Graham, M. Mushin, P. Kirkpatrick, Oxaliplatin, Nat Rev Drug Discov 3(1) (2004) 11-2.
[19] S. K. Chan, P. Du, C. Ignacio, S. Mehta, I. G. Newton, N. F. Steinmetz, Biomimetic Virus-Like Particles as Severe Acute Respiratory Syndrome Coronavirus 2 Diagnostic Tools, ACS Nano 15(1) (2021) 1259-1272.
[20] L. Lavelle, J. P. Michel, M. Gingery, The disassembly, reassembly and stability of CCMV protein capsids, J Virol Methods 146(1-2) (2007) 311-6.
[21] M. Comas-Garcia, R. F. Garmann, S. W. Singaram, A. Ben-Shaul, C. M. Knobler, W. M. Gelbart, Characterization of Viral Capsid Protein Self-Assembly around Short Single-Stranded RNA, J Phys Chem B 118(27) (2014) 7510-7519.
[22] S. Shukla, C. Wang, V. Beiss, H. Cai, T. Washington, 2nd, A. A. Murray, X. Gong, Z. Zhao, H. Masarapu, A. Zlotnick, S. Fiering, N. F. Steinmetz, The unique potency of Cowpea mosaic virus (CPMV) in situ cancer vaccine, Biomater Sci 8(19) (2020) 5489-5503.
[23] J. F. Affonso de Oliveira, S. K. Chan, A. O. Omole, V. Agrawal, N. F. Steinmetz, In Vivo Fate of Cowpea Mosaic Virus In Situ Vaccine: Biodistribution and Clearance, ACS Nano 16(11) (2022) 18315-18328.
[24] H. Zhu, Y. Shan, K. Ge, J. Lu, W. Kong, C. Jia, Oxaliplatin induces immunogenic cell death in hepatocellular carcinoma cells and synergizes with immune checkpoint blockade therapy, Cell Oncol (Dordr) 43(6) (2020) 1203-1214.
[25] J. Fucikova, O. Kepp, L. Kasikova, G. Petroni, T. Yamazaki, P. Liu, L. Zhao, R. Spisek, G. Kroemer, L. Galluzzi, Detection of immunogenic cell death and its relevance for cancer therapy, Cell Death Dis 11(11) (2020) 1013.

### REFERENCES EXAMPLE 2 (AND REFERENCES WITH SUPERSCRIPTS)

(1) Kawai, T., and Akira, S. (2010) The role of pattern-recognition receptors in innate immunity: update on Toll-like receptors. Nat Immunol 11, 373-384. Kaczanowska, S. Joseph, A. M., and Davila, E. (2013) TLR agonists: our best frenemy in cancer immunotherapy. J Leukoc Biol 93, 847-863.
(2) O'Neill, L. A., and Bowie, A. G. (2007) The family of five: TIR-domain-containing adaptors in Toll-like receptor signalling. Nat Rev Immunol 7, 353-364.
(3) Dongye, Z. Li, J., and Wu, Y. (2022) Toll-like receptor 9 agonists and combination therapies: strategies to modulate the tumour immune microenvironment for systemic anti-tumour immunity. Br J Cancer 127, 1584-1594.
(4) Cai, H. Shukla, S., and Steinmetz, N. F. (2020) The Antitumor Efficacy of CpG Oligonucleotides is Improved by Encapsulation in Plant Virus-Like Particles. Adv Funct Mater 30.
(5) Nkanga, C. I. Ortega-Rivera, O. A., and Steinmetz, N. F. (2022) Photothermal immunotherapy of melanoma using TLR-7 agonist laden tobacco mosaic virus with polydopamine coat. Nanomedicine 44, 102573.
(6) Chan, M. Hayashi, T. Mathewson, R. D. Yao, S. Gray, C. Tawatao, R. I. Kalenian, K. Zhang, Y. Hayashi, Y. Lao, F. S.; et al. (2011) Synthesis and characterization of PEGylated toll like receptor 7 ligands. Bioconjug Chem 22, 445-454.
(7) Shinchi, H.Crain, B.Yao, S.Chan, M.Zhang, S. S.Ahmadiiveli, A.Suda, Y.Hayashi, T.Cottam, H. B., and Carson, D. A. (2015) Enhancement of the Immunostimulatory Activity of a TLR7 Ligand by Conjugation to Polysaccharides. Bioconjug Chem 26, 1713-1723.
(8) Wan, D. Que, H. Chen, L. Lan, T. Hong, W. He, C. Yang, J. Wei, Y., and Wei, X. (2021) Lymph-Node-Targeted Cholesterolized TLR7 Agonist Liposomes Provoke a Safe and Durable Antitumor Response. Nano Lett 21, 7960-7969.
(9) Slater, L. Bartlett, N. W. Haas, J. J. Zhu, J. Message, S. D. Walton, R. P. Sykes, A. Dahdaleh, S. Clarke, D. L. Belvisi, M. G.; et al. (2010) Co-ordinated role of TLR3, RIG-I and MDA5 in the innate response to rhinovirus in bronchial epithelium. PLoS Pathog 6, e1001178. Yu, M., and Levine, S. J. (2011) Toll-like receptor, RIG-I-like receptors and the NLRP3 inflammasome: key modulators of innate immune responses to double-stranded RNA viruses. Cytokine Growth Factor Rev 22, 63-72.
(10) Longhi, M. P. Trumpfheller, C. Idoyaga, J. Caskey, M. Matos, I. Kluger, C. Salazar, A. M. Colonna, M., and Steinman, R. M. (2009) Dendritic cells require a systemic type I interferon response to mature and induce CD4+ Th1 immunity with poly IC as adjuvant. J Exp Med 206, 1589-1602.
(11) Mao, C. Beiss, V. Fields, J. Steinmetz, N. F., and Fiering, S. (2021) Cowpea mosaic virus stimulates antitumor immunity through recognition by multiple MYD88-dependent toll-like receptors. Biomaterials 275, 120914.
(12) Shukla, S. Wang, C. Beiss, V. Cai, H. Washington, T., 2ndMurray, A. A. Gong, X. Zhao, Z. Masarapu, H. Zlotnick, A.; et al. (2020) The unique potency of Cowpea mosaic virus (CPMV) in situ cancer vaccine. Biomater Sci 8, 5489-5503.
(13) Shukla, S. Myers, J. T. Woods, S. E. Gong, X. Czapar, A. E. Commandeur, U. Huang, A. Y. Levine, A. D., and Steinmetz, N. F. (2017) Plant viral nanoparticles-based HER2 vaccine: Immune response influenced by differential transport, localization and cellular interactions of particulate carriers. Biomaterials 121, 15-27. Lizotte, P. H. Wen, A. M. Sheen, M. R. Fields, J. Rojanasopondist, P. Steinmetz, N. F., and Fiering, S. (2016) In situ vaccination with cowpea mosaic virus nanoparticles suppresses metastatic cancer. Nat Nanotechnol 11, 295-303.
(14) Chung, Y. H. Park, J. Cai, H., and Steinmetz, N. F. (2021) S100A9-Targeted Cowpea Mosaic Virus as a Prophylactic and Therapeutic Immunotherapy against Metastatic Breast Cancer and Melanoma. Adv Sci (Weinh) 8, e2101796.
(15) Gautam, A. Beiss, V. Wang, C. Wang, L., and Steinmetz, N. F. (2021) Plant Viral Nanoparticle Conjugated with Anti-PD-1 Peptide for Ovarian Cancer Immunotherapy. Int J Mol Sci 22.
(16) Aljabali, A. A. Shukla, S. Lomonossoff, G. P. Steinmetz, N. F., and Evans, D. J. (2013) CPMV-DOX delivers. Mol Pharm 10, 3-10.
(17) Affonso de Oliveira, J. F. Chan, S. K. Omole, A. O. Agrawal, V., and Steinmetz, N. F. (2022) In Vivo Fate of Cowpea Mosaic Virus In Situ Vaccine: Biodistribution and Clearance. ACS Nano 16, 18315-18328.
(18) Chan, S. K. Du, P. Ignacio, C. Mehta, S. Newton, I. G., and Steinmetz, N. F. (2021) Biomimetic Virus-Like Particles as Severe Acute Respiratory Syndrome Coronavirus 2 Diagnostic Tools. ACS Nano 15, 1259-1272.
(19) Crooke, S. N. Schimer, J. Raji, I. Wu, B. Oyelere, A. K., and Finn, M. G. (2019) Lung Tissue Delivery of Virus-Like Particles Mediated by Macrolide Antibiotics. Mol Pharm 16, 2947-2955.
(20) Crossey, E. Amar, M. J. A. Sampson, M. Peabody, J. Schiller, J. T. Chackerian, B., and Remaley, A. T. (2015) A cholesterol-lowering VLP vaccine that targets PCSK9. Vaccine 33, 5747-5755.
(21) Rohn, T. A. Jennings, G. T. Hernandez, M. Grest, P. Beck, M. Zou, Y. Kopf, M., and Bachmann, M. F. (2006) Vaccination against IL-17 suppresses autoimmune arthritis and encephalomyelitis. Eur J Immunol 36, 2857-2867.
(22) Mohsen, M. O. Vogel, M. Riether, C. Muller, J. Salatino, S. Ternette, N. Gomes, A. C. Cabral-Miranda, G. El-Turabi, A. Ruedl, C.; et al. (2019) Targeting Mutated Plus Germline Epitopes Confers Pre-clinical Efficacy of an Instantly Formulated Cancer Nano-Vaccine. Front Immunol 10, 1015. Wu, X. Yin, Z. McKay, C. Pett, C. Yu, J. Schorlemer, M. Gohl, T. Sungsuwan, S. Ramadan, S. Baniel, C.; et al. (2018) Protective Epitope Discovery and Design of MUC1-based Vaccine for Effective Tumor Protections in Immunotolerant Mice. J Am Chem Soc 140, 16596-16609. Wu, X. McKay, C. Pett, C. Yu, J. Schorlemer, M. Ramadan, S. Lang, S. Behren, S. Westerlind, U. Finn, M. G.; et al. (2019) Synthesis and Immunological Evaluation of Disaccharide Bearing MUC-1 Glycopeptide Conjugates with Virus-like Particles. ACS Chem Biol 14, 2176-2184.
(23) Cheng, Y. Lemke-Miltner, C. D. Wongpattaraworakul, W. Wang, Z. Chan, C. H. F. Salem, A. K. Weiner, G. J., and Simons, A. L. (2020) In situ immunization of a TLR9 agonist virus-like particle enhances anti-PD1 therapy. J Immunother Cancer 8,
(24) Ali, A., and Roossinck, M. J. (2007) Rapid and efficient purification of Cowpea chlorotic mottle virus by sucrose cushion ultracentrifugation. J Virol Methods 141, 84-86. Wen, A. M.Lee, K. L. Yildiz, I. Bruckman, M. A. Shukla, S., and Steinmetz, N. F. (2012) Viral nanoparticles for in vivo tumor imaging. J Vis Exp, e4352.
(25) Leong, H. S. Steinmetz, N. F. Ablack, A. Destito, G. Zijlstra, A. Stuhlmann, H. Manchester, M., and Lewis, J. D. (2010) Intravital imaging of embryonic and tumor neovasculature using viral nanoparticles. Nat Protoc 5, 1406-1417.
(26) Ortega-Rivera, O. A. Pokorski, J. K., and Steinmetz, N. F. (2021) A single-dose, implant based, trivalent virus-like particle vaccine against “cholesterol checkpoint” proteins. Adv Ther (Weinh) 4,
(27) Momin, N. Palmeri, J. R. Lutz, E. A. Jailkhani, N. Mak, H. Tabet, A. Chinn, M. M. Kang, B. H. Spanoudaki, V. Hynes, R. O.; et al. (2022) Maximizing response to intratumoral immunotherapy in mice by tuning local retention. Nat Commun 13, 109. Jeganathan, S. Budziszewski, E. Hernandez, C. Dhingra, A., and Exner, A. A. (2020) Improving Treatment Efficacy of In Situ Forming Implants via Concurrent Delivery of Chemotherapeutic and Chemosensitizer. Sci Rep 10, 6587.
(28) Koellhoffer, E. C., and Steinmetz, N. F. (2022) Cowpea Mosaic Virus and Natural Killer Cell Agonism for In Situ Cancer Vaccination. Nano Lett 22, 5348-5356.
(29) Wang, C., and Steinmetz, N. F. (2020) A Combination of Cowpea Mosaic Virus and Immune Checkpoint Therapy Synergistically Improves Therapeutic Efficacy in Three Tumor Models. Adv Funct Mater 30,
(30) Hoopes, P. J. Wagner, R. J. Duval, K. Kang, K. Gladstone, D. J. Moodie, K. L. Crary-Burney, M. Ariaspulido, H. Veliz, F. A. Steinmetz, N. F.; et al. (2018) Treatment of Canine Oral Melanoma with Nanotechnology-Based Immunotherapy and Radiation. Mol Pharm 15, 3717-3722.
(31) Ghani, M. A. Bangar, A. Yang, Y. Jung, E. Sauceda, C. Mandt, T. Shukla, S. Webster, N. J. G. Steinmetz, N. F., and Newton, I. G. (2023) Treatment of Hepatocellular Carcinoma by Multimodal In Situ Vaccination Using Cryoablation and a Plant Virus Immunostimulant. J Vase Interv Radiol.
(32) Cai, H. Wang, C. Shukla, S., and Steinmetz, N. F. (2019) Cowpea Mosaic Virus Immunotherapy Combined with Cyclophosphamide Reduces Breast Cancer Tumor Burden and Inhibits Lung Metastasis. Adv Sci (Weinh) 6, 1802281.

What is claimed is:

1. A method for inhibiting the growth of a mammalian cancer cell growth or stimulating the immune response of a mammalian cell, comprising contacting the cell with an effective amount of a viral nanoparticle comprising at least one TLR agonist selected from a double-stranded RNA (dsRNA) or a synthetic dsRNA that activates a TLR and a chemotherapeutic agent, thereby inhibiting the growth of the mammalian cancer cell or stimulate the immune response of the mammalian cell.

2. A method for one or more of:
(a) treating cancer in a subject in need thereof,
(b) inhibiting, delaying, slowing down, or preventing relapse of cancer in a subject in need thereof, or
(c) triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells,
comprising administering to the subject administering an effective amount of a viral nanoparticle, wherein the nanoparticle comprises at least one TLR agonist selected from a double-stranded RNA (dsRNA) or a synthetic dsRNA capable of activating a TLR; and a chemotherapeutic agent, and
thereby treating the cancer, delaying, slowing down, or preventing the relapse of cancer, or triggering or enhancing one or more of the following in a subject in need thereof: an immune response, immunostimulatory cytokines, an increase in the ratio of M1 (antitumor) to M2 (protumor) macrophages, an increase in natural killer cells (NK cells), an increase in CD4+ cells, or an increase in CD8+ cells in the subject in the subject.

3. The method of claim 2, wherein the immunostimulatory cytokine or chemokine is selected from IL-4, IL-5, IL-13, IL-15, IFN-γ, IP-10, MCP-1, MCP-1α, MCP-1β, Eotoxin, or TNF-α.

4. The method of claim 1, wherein the viral nanoparticle is derived from a Cowpea chlorotic mottle virus (CCMV), a Cowpea mosaic virus (CPMV), a Physalis mottle virus (PhMV), a Papaya mosaic virus (PapMV), a Potato virus X (PVX), or a bacteriophage Qbeta (Qβ).

5. The method of claim 1, wherein the nanoparticle comprises a capsid protein, further optionally from CCMV, CPMA compositions and methods may a method for inhibiting the growth of a mammalian cancer cell growth or stimulating the immune response of a mammalian cell, comprising contacting the cell with an effective amount of a viral nanoparticle comprising at least one TLR agonist selected from a double-stranded RNA (dsRNA) or a synthetic dsRNA that activates a TLR and a chemotherapeutic agent, thereby inhibiting the growth of the mammalian cancer cell or stimulate the immune response of the mammalian cell. V, or a combination thereof and further optionally wherein the TLR is TLR3, optionally wherein the TLR3 agonist is poly(I:C), and yet further optionally wherein the chemotherapeutic agent is selected from 5-fluorouracil, methyl-CCNU, oxaliplatin, irinotecan, mitomycin, cytarabine, doxorubicin, or cyclophosphamide.

6. The method of claim 5, wherein the chemotherapeutic agent comprises or consists essentially of oxaliplatin.

7. A composition comprising (a) a viral nanoparticle comprising at least one toll-like receptor (TLR) agonist selected from a double-stranded RNA (dsRNA) or a synthetic dsRNA capable of activating a TLR; and (b) at least one chemotherapeutic agent, and optionally a carrier, and further optionally a detectable label, wherein the TLR is selected from TLR1, TLR2, TLR3, TLR4, TLR5, TLR6, TLR7, TLR8, or TLR9, optionally wherein the virus is CPMV, CMMV, or Qβ.

8. The composition of claim 7, wherein the chemotherapeutic agent is selected from 5-fluorouracil, methyl-CCNU, oxaliplatin, irinotecan, mitomycin, cytarabine, doxorubicin, or cyclophosphamide, and optionally wherein the chemotherapeutic agent comprises or consists essentially of oxaliplatin.

9. The composition of claim 7, wherein the TLR is TLR3, the TLR agonist is polyinosinic:polycytidylic acid (poly(I:C)), optionally wherein the chemotherapeutic agent comprises or consists essentially of oxaliplatin.

10. A method to inhibit the growth of a mammalian cancer cell or stimulate the immune response of a mammalian cell, comprising contacting the cell the composition of claim 7, thereby inhibiting the growth of the cancer cell or stimulating the immune response of a mammalian cell.

11. A method for:
(a) treating cancer in a subject in need thereof,
(b) inhibiting, delaying, slowing down, or preventing relapse of cancer in a subject in need thereof, or
(c) triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells, comprising administering to the subject an effective amount of the composition of claim 7, thereby treating the cancer, delaying, slowing down, or preventing the relapse of cancer, or triggering or enhancing one or more of the following in a subject in need thereof: an immune response, immunostimulatory cytokines, an increase in the ratio of M1 (antitumor) to M2 (protumor) macrophages, an increase in natural killer cells (NK cells), an increase in CD4+ cells, or an increase in CD8+ cells in the subject in the subject.

12. A composition comprising (a) a viral nanoparticle comprising a toll-like receptor (TLR) agonist capable of activating a TLR; and (b) a chemotherapeutic agent, and optionally a carrier, and further optionally a detectable label, wherein the TLR is selected from TLR1, TLR2, TLR3, TLR4, TLR5, TLR6, TLR7, TLR8, or TLR9, optionally wherein the virus is CPMV, CMMV, or Qβ, and the chemotherapeutic agent is optionally selected from 5-fluorouracil, methyl-CCNU, oxaliplatin, irinotecan, mitomycin, cytarabine, doxorubicin, or cyclophosphamide.

13. The composition of claim 12, wherein the TLR is TLR7, wherein the TLR agonist is 2-methoxyethoxy-8-oxo-9-(4-carboxybenzyl)adenine (1V209), optionally wherein the chemotherapeutic agent comprises or consists essentially of oxaliplatin, and further optionally wherein the TLR is TLR3, the TLR agonist is polyinosinic:polycytidylic acid (poly(I:C)), optionally wherein the chemotherapeutic agent comprises or consists essentially of oxaliplatin.

14. A method to prevent mammalian cell growth or stimulating the immune response of a mammalian cell comprising contacting the cell the composition of claim 12, thereby preventing the mammalian cell growth or stimulating the immune response of the mammalian cell.

15. A method for:
(a) treating cancer in a subject in need thereof,
(b) inhibiting, delaying, slowing down, or preventing relapse of cancer in a subject in need thereof, or
(c) triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells,
comprising administering to the subject an effective amount of the composition of claim 12, and thereby treating the cancer, delaying, slowing down, or preventing the relapse of cancer, or triggering or enhancing one or more of the following in a subject in need thereof: an immune response, immunostimulatory cytokines, an increase in the ratio of M1 (antitumor) to M2 (protumor) macrophages, an increase in natural killer cells (NK cells), an increase in CD4+ cells, or an increase in CD8+ cells in the subject in the subject.

16. A composition comprising (a) a viral nanoparticle comprising at least two toll-like receptor (TLR) agonists capable of activating a TLR; and (b) at least one chemotherapeutic agent, and optionally a carrier, and further optionally a detectable label, wherein the TLR is selected from TLR1, TLR2, TLR3, TLR4, TLR5, TLR6, TLR7, TLR8, or TLR9, optionally wherein the plant virus is CPMV, CMMV, or Qβ, and optionally wherein the at least two TLR agonists activate the same TLR or different TLRs.

17. The composition of claim 16, wherein the at least two TLR agonists are 1V209 and poly(I:C).

18. The composition of claim 16, wherein the chemotherapeutic agent is selected from 5-fluorouracil, methyl-CCNU, oxaliplatin, irinotecan, mitomycin, cytarabine, doxorubicin, cyclophosphamide.

19. A method to prevent mammalian cell growth or stimulating the immune response of a mammalian cell comprising contacting the cell the composition of claim 16, thereby preventing the mammalian cell growth or stimulating the immune response of the mammalian cell.

20. A method for:
(a) treating cancer in a subject in need thereof;
(b) inhibiting, delaying, slowing down, or preventing relapse of cancer in a subject in need thereof; or
(c) triggering or enhancing one or more of the following in a subject in need thereof: an immune response, immunostimulatory cytokines, chemokines, an increase in the ratio of M1 (antitumor) to M2 (protumor) macrophages, an increase in natural killer cells (NK cells), an increase in CD4+ cells, or an increase in CD8+ cells,
comprising administering to the subject an effective amount of the composition of claim 16, and
thereby treating the cancer, delaying, slowing down, or preventing the relapse of cancer, or triggering or enhancing one or more of the following in a subject in need thereof: an immune response, immunostimulatory cytokines, an increase in the ratio of M1 (antitumor) to M2 (protumor) macrophages, an increase in natural killer cells (NK cells), an increase in CD4+ cells, or an increase in CD8+ cells in the subject in the subject, and optionally wherein the immunostimulatory cytokine or chemokine is selected IL-4, IL-5, IL-13, IL-15, IFN-γ, IP-10, MCP-1, MCP-1α, MCP-1β, Eotoxin, or TNF-α.


SEQUENCE LISTING (4 sequences)

SEQ ID NO: 1 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 2 - protein, 374 aa, Cowpea mosaic virus
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 3 - protein, 189 aa, Cowpea mosaic virus
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 4 - protein, 133 aa, Qubevirus durum
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY